(19) United States
(12) Patent Application Publication
(10) Pub. No.: US 2009/0228996 A1
(43) Pub. Date: Sep. 10, 2009

(54) Means and Methods for influencing Interactions Between Dc-Sign and Dc-Sign Ligands

(76) Inventors: William Anderson Paxton, Amsterdam (NL); Georgios Pollakis, Amsterdam (NL); Marloes Albertine Naarding, Amsterdam (NL)

Correspondence Address: TRASKBRITT, P.C., P.O. BOX 2550, SALT LAKE CITY, UT, 84110, US

(21) Appl. No.: 12/223,626

(22) PCT Filed: Feb. 3, 2006
(86) PCT No.: PCT/NL2006/000050

§ 371 Date: Feb. 2, 2009

(51) Int. Cl. A01K 67/00; G01N 33/566; C12Q 1/34; A61K 38/46; A61K 39/00

(52) U.S. Cl. 800/13; 436/501; 435/18; 424/94.6; 424/184.1

(57) ABSTRACT

The invention provides a method for influencing interaction between a DC-SIGN ligand receptor that is exposed to said DC-SIGN ligand, comprising at least one Lewis X sugar epitope, or a functional part, derivative and/or analogue thereof, and a non-saccharide moiety. One preferred embodiment provides a method according to the invention, wherein said compound comprises polyacrylamide-Lewis X, bovine serum albumin-Lewis X and/or bile salt stimulated lipase (BSSL), or a functional part, derivative and/or analogue

The invention relates to the fields of molecular biology and immunology. More specifically, the invention relates to receptor-ligand interaction.

Dendritic cells (DCs), being antigen presenting cells, play an important role in the immune system of animals. Interactions between DCs and surrounding compounds occur frequently. DCs express, among other receptors, the DC specific ICAM-3 grabbing non-integrin (DC-SIGN) receptor which is a C-type lectin containing an external calcium-dependent mannose binding lectin domain. DC-SIGN interacts with a variety of compounds such as for instance the envelope glycoprotein gp120 of human immunodeficiency virus type 1 (HIV-1), HIV-2 and simian immunodeficiency virus (SIV) as well as other pathogens such as hepatitis C, Ebola, cytomegalovirus, Dengue virus, Mycobacterium, Leishmania, Candida albicans and Helicobacter pylori. DC-SIGN has been implicated to play an important role in pathogen transmission and the establishment of infection. For instance, the interaction of HIV with DC-SIGN can lead to infection of the DCs or alternatively the virus can be internalized into a trypsin resistant compartment prior to transfer to its main target cell, resulting in enhancement of infection of CD4+ T-lymphocytes.

The DC-SIGN receptor is also capable of binding ICAM 2 and ICAM3. ICAM2 is expressed on endothelial cells and ICAM 3 is expressed on T cells. DC-SIGN furthermore interacts with β2-integrin Mac-1 (CD11b/CD18), which is expressed on neutrophils and promotes the interaction with DC cells, therefore controlling the immune responses mounted. As another example, CEACAM1, which is expressed on neutrophils is also capable of interacting with DC-SIGN.

Interactions between DC-SIGN and DC-SIGN ligands often have far-reaching consequences which are often beneficial to the host, for instance when an infection is effectively cleared. At other occasions however, interactions between DC-SIGN and a DC-SIGN ligands have adverse effects, such as for instance HIV infection of DCs and subsequent infection of CD4+ T-lymphocytes. It would therefore be advantageous to be capable of counteracting such undesired interactions between DC-SIGN and DC-SIGN ligands, whereas it would also be desirable to enhance desired, beneficial interactions between DC-SIGN and DC-SIGN ligands.

It is an object of the present invention to provide means and methods for influencing interactions between DC-SIGN and DC-SIGN ligands.

In one aspect the invention provides a method for influencing interaction between a DC-SIGN ligand and a DC-SIGN receptor that is exposed to said DC-SIGN ligand, comprising regulating interaction between said DC-SIGN receptor and a compound comprising at least one Lewis X sugar epitope, or a functional part, derivative and/or analogue thereof, and a non-saccharide moiety.

According to the invention, a compound comprising at least one Lewis X sugar epitope, or a functional part, derivative and/or analogue thereof, and a non-saccharide moiety is capable of specifically binding a DC-SIGN receptor. Binding of a compound according to the invention to a DC-SIGN receptor at least partly inhibits interaction between said DC-SIGN receptor with other DC-SIGN ligands. Hence, interaction between a DC-SIGN receptor and a DC-SIGN ligand is influenced by regulating interaction between said DC-SIGN receptor and a compound according to the invention which comprises at least one Lewis X sugar epitope, or a functional part, derivative and/or analogue thereof, and a non-saccharide moiety.

A DC-SIGN ligand is defined herein as a compound capable of specifically binding DC-SIGN. Non-limiting examples of DC-SIGN ligands are HIV gp120 proteins and HCV envelope glycoproteins. As will be recognized, various DC-SIGN ligands are often capable of binding DC-SIGN to a different extent.

A Lewis X (Le^{x}) sugar epitope comprises 3-fucosyl-N-acetyllactosamine. Le^{x} is a sugar epitope that is a member of the Lewis group antigens, which can be part of a larger oligosaccharide either associated with other moieties or not. The sugar epitope has been identified in many bodily fluids, including saliva, blood and human milk and has been shown to be present in a number of pathogens as well as pathogen extracts.

A functional part of a Lewis X sugar epitope is defined as a part which has at least one same property as a Lewis X sugar epitope in kind, not necessarily in amount. Said functional part, when bound to a non-saccharide moiety, is preferably capable of binding DC-SIGN.

A functional derivative of a Lewis X sugar epitope is defined as a Lewis X sugar epitope which has been altered such that at least one property—preferably a DC-SIGN-binding property—of the resulting compound is essentially the same in kind, not necessarily in amount.

A person skilled in the art is well able to generate analogous compounds of a Lewis X sugar epitope. Such an analogue has essentially at least one same property—preferably a DC-SIGN-binding property—as said Lewis X sugar epitope in kind, not necessarily in amount.

As used herein, the term “Lewis X sugar epitope” also encompasses a functional part, derivative and/or analogue of a Lewis X sugar epitope, unless expressly stated otherwise.

A compound of the invention comprising at least one Lewis X sugar epitope, or a functional part, derivative and/or analogue thereof, furthermore comprises a non-saccharide moiety. A non-saccharide moiety is defined as a moiety comprising at least a non-saccharide part. Preferably, said non-saccharide moiety comprises a proteinaceous moiety and/or a polymer moiety. A Lewis X sugar epitope, or a functional part, derivative and/or analogue thereof, bound to a proteinaceous moiety and/or a polymer moiety is particularly suitable for binding DC-SIGN.

A DC-SIGN receptor is exposed to a DC-SIGN ligand if said DC-SIGN receptor and said DC-SIGN ligand are, in principle, capable of approaching each other to such extent that they are capable of interacting when no other (competing) compounds are present.

By influencing interaction between a DC-SIGN ligand and a DC-SIGN receptor that is exposed to said DC-SIGN ligand is meant herein that the capability of said DC-SIGN ligand and said DC-SIGN receptor of interacting with each other is altered. Preferably, their capability of binding is influenced. This is preferably performed by providing said receptor with a compound of the invention. Said compound of the invention is preferably capable of competing with another DC-SIGN ligand for a binding site of DC-SIGN. In one preferred embodiment binding of said receptor and said ligand is at least in part counteracted, in order to at least in part counteract an adverse effect of an interaction between a DC-SIGN receptor and a DC-SIGN ligand.

By regulating interaction between said DC-SIGN receptor and a compound comprising at least one Lewis X sugar epitope, or a functional part, derivative and/or analogue thereof, and a non-saccharide moiety is meant herein that an interaction between said DC-SIGN receptor and said compound is at least in part induced, altered and/or inhibited. One preferred embodiment comprises inducing and/or enhancing an interaction between a compound according to the invention and a DC-SIGN receptor, in order to at least in part prevent binding of said DC-SIGN receptor to another DC-SIGN ligand.

An interaction between a DC-SIGN receptor and a DC-SIGN ligand is influenced in various ways. Preferably, said interaction is regulated by regulating the amount and/or activity of a compound according to the invention in an environment wherein said DC-SIGN receptor is exposed to said compound. If the amount and/or activity of a compound according to the invention is enhanced, said compound according to the invention will be better capable of counteracting interaction between DC-SIGN and another DC-SIGN ligand. If the amount and/or activity of a compound according to the invention is diminished, said compound according to the invention will be less capable of counteracting interaction between DC-SIGN and another DC-SIGN ligand. As a result, interaction between said other DC-SIGN ligand and a DC-SIGN receptor is enhanced. Hence, interaction between a DC-SIGN ligand and a DC-SIGN receptor is regulated by regulating the amount and/or activity of a compound according to the present invention in an environment wherein said DC-SIGN receptor is exposed to said compound. Preferably provided is therefore a method according to the invention wherein an interaction between said DC-SIGN receptor and said compound comprising at least one Lewis X sugar epitope, or a functional part, derivative and/or analogue thereof, and a non-saccharide moiety is regulated by regulating the amount and/or activity of said compound in an environment wherein said DC-SIGN receptor is exposed to said compound.

By the activity of a compound according to the invention is meant herein the capability of said compound of interacting with a DC-SIGN receptor. An activity of a compound according to the present invention is for instance diminished by providing a compound according to the invention with a second compound which is capable of specifically binding said compound according to the invention, thereby at least partly influencing the DC-SIGN affinity of said compound according to the present invention (for instance, but not limited to, by at least partly shielding a DC-SIGN binding site and/or altering the conformation of a compound according to the invention). An activity of a compound according to the present invention is also influenced by altering a compound to the invention such that its capability of binding DC-SIGN is altered, for instance via genetic engineering and/or conventional synthesis and/or modification techniques, which are well known in the art.

One preferred embodiment provides a method according to the invention, wherein said compound comprises polyacrylamide-Lewis X, bovine serum albumin-Lewis X and/or bile salt stimulated lipase (BSSL), or a functional part, derivative and/or analogue thereof. According to the invention, these compounds are particularly suitable for binding DC-SIGN, and, hence, particularly suitable for influencing interaction between DC-SIGN and other DC-SIGN ligands. Polyacrylamide-Lewis X and bovine serum albumin-Lewis X are artificial compounds wherein Lewis X is coupled to a polyacrylamide (PAA) backbone and a bovine serum albumin (BSA) backbone, respectively. Bile salt stimulated lipase is a compound which is endogenously present in animals. Bile salt stimulated lipase is a glycoprotein secreted by the pancreas and activated by bile salts in the intestine. The protein is also known as Bile Salt Dependent Lipase (BSDL), Carboxyl Ester Lipase (CEL), lysophospholipase and cholesterol esterase. It possesses a broad substrate specificity that contributes to the hydrolysis of dietary mono-, di- and tri-acylglycerols and is responsible for digestion of fat-soluble vitamin esters and cholesterol esters in the small intestine. BSSL is also expressed by the mammary gland and is present in human milk at a concentration of about 100-200 μg/ml. Neonates normally only secrete small amounts of pancreatic lipase into the duodenum so gastric lipase as well as BSSL in human milk markedly enhance fat digestion in the newborn. Both pancreatic and human milk BSSL have been shown to be identical in amino acid sequence, but differ substantially in their carbohydrate content with Le^{x} having been shown to be present at the C-terminus of BSSL.

According to the present invention, BSSL, or a functional part, derivative and/or analogue thereof, is capable of binding a DC-SIGN receptor. This way, BSSL is capable of influencing interaction between a DC-SIGN receptor and other DC-SIGN ligands. Hence, interaction between a DC-SIGN receptor and a DC-SIGN ligand is influenced by regulating interaction between a DC-SIGN receptor and BSSL or a functional part, derivative and/or analogue of BSSL. For instance, administration of BSSL or a functional part, derivative and/or analogue thereof at least in part counteracts interaction between a DC-SIGN receptor and other DC-SIGN ligands.

A functional part of BSSL is defined as a part which has at least one same property as BSSL in kind, not necessarily in amount. Said functional part is preferably capable of binding DC-SIGN. A functional derivative of BSSL is defined as a BSSL which has been altered such that at least one property—preferably a DC-SIGN-binding property—of the resulting compound is essentially the same in kind, not necessarily in amount. A derivative is provided in many ways, for instance through, but not limited to, conservative amino acid substitution, by altering the amount of proline rich repeat units in the carboxyl-terminal region of BSSL, and/or by altering the number of O-linked glycosylations in at least one proline rich repeat unit in the carboxyl-terminal region of BSSL.

A person skilled in the art is well able to generate analogous compounds of BSSL. Such an analogue has essentially at least one same property—preferably a DC-SIGN-binding property—as BSSL in kind, not necessarily in amount.

As used herein, the term “BSSL” also encompasses a functional part, derivative and/or analogue of BSSL, unless expressly stated otherwise.

The use of BSSL is preferred, since this is a compound endogenously present in animals. Administration of a BSSL variant which is essentially similar, preferably identical, to endogenous BSSL is preferred since a lower immune response will be elicited, if any. Furthermore, it is for instance possible to diminish the amount and/or activity of endogenously present BSSL in order to enhance interaction between DC-SIGN and other DC-SIGN ligands. Hence, the insight of the present invention that BSSL is capable of interacting with DC-SIGN provides novel and favourable applications which were not possible before the present invention. One particularly preferred embodiment therefore provides a method for influencing interaction between a DC-SIGN ligand and a DC-SIGN receptor that is exposed to said DC-SIGN ligand, comprising regulating interaction between said DC-SIGN receptor and BSSL, or a functional part, derivative and/or analogue thereof. In one embodiment, the amount and/or activity of BSSL in an animal is increased in order to counteract interaction between DC-SIGN receptors and other DC-SIGN ligands. The amount of BSSL is for instance increased by administering BSSL, or a functional part, derivative, analogue and/or precursor thereof, to an animal.

A precursor of BSSL is defined as a compound which is converted into BSSL when said compound is processed in vivo. After administration of a given compound to an animal, said compound is sometimes altered within said animal. Said compound is for instance cleaved. Alternatively, or additionally, said compound, or a metabolite thereof, is for instance modified by glycosylation. Various other modifications are possible in vivo. As a result, a BSSL precursor is modified in vivo such that BSSL is formed.

In one embodiment an animal is provided with a nucleic acid sequence encoding BSSL. Expression of said nucleic acid results in an enhanced BSSL level. Methods for producing a nucleic acid construct encoding BSSL, as well as methods for administering such construct to an animal, are known in the art. For instance, a (retro)viral vector is used. Preferably, said nucleic acid encoding BSSL is operably linked to a tissue-specific promoter, so that BSSL expression is restricted to (a) particular site(s) of interest.

The invention furthermore provides the insight that various isoforms of BSSL are particularly suitable for influencing interaction between a DC-SIGN ligand and a DC-SIGN receptor. Various isoforms of BSSL are present in animals due to, amongst other things, the number of proline-rich repeats at the C-terminus, the number of O-linked glycosylations in said repeats and/or variation in O-linked glycosylation sites of differential post-translational modifications, for instance due to differences in sugar processing machineries between individuals (such as for instance differential expression of fucosyltransferases). According to the present invention, a human BSSL isoform of between 90 and 115 kDa, preferably between 95 and 110 kDa, most preferably about 102 kDa is particularly suitable for influencing interaction between a DC-SIGN ligand and a DC-SIGN receptor. Furthermore, a human BSSL isoform having between 13-16, preferably about 14, proline-rich repeat units in its carboxyl-terminal region is preferred since it is particularly suitable for influencing interaction between a DC-SIGN ligand and a DC-SIGN receptor. The invention therefore provides a method according to the invention, wherein said compound comprises a human BSSL isoform of between 90 and 115 kDa, preferably between 95 and 110 kDa, most preferably about 102 kDa, or a functional part, derivative and/or analogue thereof.

In one particularly preferred embodiment said compound comprises a human BSSL isoform having between 13-16, preferably about 14, proline-rich 11-amino acid repeat units in its carboxyl-terminal region, or a functional part, derivative and/or analogue of said BSSL isoform.

In case of a risk of an undesired binding event between a DC-SIGN receptor and a DC-SIGN ligand, a compound according to the invention is preferably used in order to counteract such binding event. In such case said binding event is preferably at least in part prevented. For instance, in case of a risk of binding of a pathogen to DC-SIGN, the amount and/or activity of a compound according to the invention, preferably BSSL, is preferably increased in order to counteract binding of said pathogen. One embodiment thus provides a method according to the invention wherein binding of a DC-SIGN receptor to a DC-SIGN ligand is at least in part counteracted. A DC-SIGN receptor is preferably bound to a compound according to the invention, preferably BSSL, in order to at least in part prevent binding of said DC-SIGN receptor to another, undesired DC-SIGN ligand such as a pathogen capable of binding DC-SIGN.

Interaction between DC-SIGN and an undesired DC-SIGN ligand is preferably counteracted by increasing the amount of a compound according to the invention. Said compound preferably competes with said undesired ligand for DC-SIGN, thereby at least in part preventing interaction between said undesired ligand and DC-SIGN. The amount of a compound according to the invention is in one embodiment increased by administering said compound to an individual in need thereof. Preferred compounds according to the invention are polyacrylamide-Lewis X, bovine serum albumin-Lewis X and bile salt stimulated lipase (BSSL). A method according to the invention comprising administering polyacrylamide-Lewis X, bovine serum albumin-Lewis X, bile salt stimulated lipase (BSSL), or a functional part, derivative and/or analogue thereof, to a DC-SIGN receptor is therefore herewith provided. Most preferably, BSSL (or a functional part, derivative and/or analogue thereof) is administered. Said BSSL is preferably essentially similar to, most preferably identical to, endogenous BSSL in order to at least in part avoid an immune response. If a compound according to the invention is administered to an individual, it is preferably present in a composition such as for instance a food product, or a pharmaceutical composition comprising a pharmaceutically acceptable carrier, diluent and/or excipient, in order to enhance administration.

In one preferred embodiment, the amount of BSSL is increased by providing an individual with a nucleic acid sequence encoding (exogenous) BSSL, and/or by upregulating expression of endogenous BSSL in an individual.

Upregulation of endogenous BSSL is in one embodiment performed by producing a non-human transgene animal wherein a stronger promoter has been operably linked to its endogenous BSSL gene. In one preferred embodiment said promoter comprises an αS1-casein or β-casein promoter. Methods for producing non-human transgene animals are well known in the art. Such non-human animal is for instance particularly suitable for research purposes. Moreover, such non-human animal is particularly suitable for producing BSSL. In one embodiment BSSL produced by said non-human animal is harvested and preferably used for human prophylactic and/or therapeutic applications. One embodiment therefore provides a method according to the invention, comprising upregulating expression of BSSL in an individual comprising said DC-SIGN receptor. A non-human animal comprising an exogenous nucleic acid sequence encoding BSSL and/or a functional part, derivative and/or analogue thereof is also herewith provided, as well as a non-human animal comprising an (exogenous) compound capable of enhancing expression and/or activity of BSSL or a functional part, derivative and/or analogue thereof.

A method according to the invention is suitable for influencing a wide variety of interactions between DC-SIGN receptors and DC-SIGN ligands. In one embodiment a method according to the invention is applied in order to counteract binding of a pathogen to a DC-SIGN receptor. One important application of a method according to the invention is at least partial prevention of infection of an individual by a micro organism which is capable of binding a DC-SIGN receptor. Micro organisms capable of binding DC-SIGN are capable of binding dendritic cells. Binding of antigens to dendritic cells and presentation of antigens to T cells are important events in cellular as well as humoral immune responses. However, binding and infection of dendritic cells by pathogens which cannot be eliminated by an individual's immune system is preferably avoided.

Once an individual has been infected by a micro organism, dendritic cells play a role in the spreading of the micro organism within said individual. Once a pathogen is bound to dendritic cells, it is internalized and taken to the lymph nodes, where it is exposed to other immune cells. When an individual is unable to clear the infection, the pathogen will spread. For instance, in case of HIV, once HIV has been captured by dendritic cells at the mucosal surface they are internalized and taken to the lymph nodes. Within the lymph nodes the dendritic cells interact with CD4 lymphocytes and transfer HIV. Subsequently, dendritic cells present in lymph nodes are capable of capturing HIV via their DC-SIGN receptor and efficiently presenting virus to other T cells. This amplifies a reinfection process manifold and contributes to amplification and spread of HIV within a patient. Hence, dendritic cells amplify an HIV infection of CD4 lymphocytes in vivo. Counteracting interaction between dendritic cells and HIV therefore lowers viral load.

Hence, infection as well as spread of a micro organism which is capable of binding DC-SIGN is preferably counteracted by at least in part preventing binding of said micro organism to DC-SIGN with a method according to the invention. Provided is therefore a method for at least in part counteracting infection of an individual by a micro organism capable of binding a DC-SIGN receptor, and/or at least in part counteracting spread of a micro organism capable of binding a DC-SIGN receptor in an individual, comprising:

providing said individual with a compound comprising at least one Lewis X sugar epitope, or a functional part, derivative and/or analogue thereof, and a non-saccharide moiety, and/or

increasing the amount and/or activity of a compound comprising at least one Lewis X sugar epitope, or a functional part, derivative and/or analogue thereof, and a non-saccharide moiety in said individual.

Non-limiting examples of pathogens known to be capable of binding DC-SIGN are HIV-1, HIV-2, SIV, hepatitis C, Ebola, cytomegalovirus, Dengue virus, human herpes virus 8, Mycobacterium, Helicobacter pylori, Leishmania and Candida albicans. A method according to the invention wherein said DC-SIGN ligand comprises HIV-1, HIV-2, SIV, hepatitis C, Ebola, cytomegalovirus, Dengue virus, human herpes virus 8, Mycobacterium, Helicobacter pylori, Leishmania and/or Candida albicans is therefore also herewith provided. Most preferably, said ligand comprises HIV. As shown in the examples, a compound according to the invention, preferably BSSL (or a functional part, derivative and/or analogue thereof), is particularly suitable for counteracting binding of HIV to DC-SIGN and subsequent transfer of HIV to CD4+ lymphocytes. HIV is capable of binding a DC-SIGN receptor via its gp120 protein. Therefore, in order to counteract HIV infection and/or spread of HIV in an infected individual, a method according to the invention preferably comprises counteracting interaction between DC-SIGN and a gp120 protein.

As another non-limiting example, HCV is capable of binding DC-SIGN via envelope glycoproteins. Therefore, in order to at least in part prevent HCV infection and/or spread of HCV, one embodiment provides a method according to the invention wherein interaction between DC-SIGN and a HCV envelope glycoprotein is at least in part prevented.

As stated herein before, a compound according to the invention preferably comprises bile salt stimulated lipase (BSSL) or a functional part, derivative and/or analogue thereof since this compound is endogenously present in animals.

In one preferred embodiment a compound according to the invention is administered to an individual. Common routes of administration are suitable, dose ranges of compounds according to the invention to be used in prophylactic and/or therapeutic applications according to the invention are designed on the basis of rising dose studies in the clinic in clinical trials for which rigorous protocol requirements exist. In one preferred embodiment a compound according to the invention is present in a microbicide, which is defined herein as an agent capable of at least in part preventing infection with micro organisms and/or capable of at least in part destroying micro organisms. Preferably a topical microbicide is used which is suitable for applying to mucosal surfaces in order to act as a barrier against infection. A microbicide according to the invention is preferably used in order to prevent infection and/or spread of sexually transmitted micro organisms such as for instance HIV, cytomegalovirus and human herpes virus. Most preferably, infection and spread of HIV is prevented.

In another embodiment an individual is provided with oral prophylaxis comprising a compound according to the invention. Various kinds of products are suitable. In one embodiment a composition is used comprising a compound according to the present invention and a suitable carrier, diluent and/or excipient. Said composition preferably comprises a pharmaceutical composition and/or a prophylactic agent.

In one preferred embodiment a compound according to the invention, preferably BSSL or a functional part, derivative and/or analogue thereof, is provided to a food product. Since BSSL is naturally present in the gastrointestinal tract, this route of administration most closely mimics the natural situation. Any kind of food product which does not completely destroy or inactivate a compound according to the invention is suitable for this purpose. In one embodiment a solid food product, such as for instance candy, is provided with a compound according to the invention. In another embodiment a compound of the invention is added to a fluid product.

In a particularly preferred embodiment a compound according to the invention, preferably BSSL, is administered to milk. Preferably mother's milk is provided with a compound according to the present invention. This is preferably performed in order to counteract mother to child transmission of undesired pathogens. Mother to child transmission (MTCT) of pathogens such as for instance HIV can occur in utero, intrapartum and by breastfeeding, with breastfeeding accounting for a significant amount of all MTCTs cases. Milk from an infected mother contains potentially infected macrophages, lymphocytes, DCs but also free pathogen. Relatively little is known with regard to the route of transmission through breastfeeding, however for the child to become infected a pathogen has to cross the mucosal barrier or be introduced to the lymphoid tissues by a breach in the epithelial layer. DCs expressed in the tonsils, upper rim of the esophagus or the intestinal tract heighten pathogen capture and transmission. In order to counteract mother to child transmission, mother's milk is preferably provided with a compound of the invention, which is capable of at least in part counteracting binding of pathogens to dendritic cells of an infant. This way, infection of an infant is at least in part prevented. Preferably, BSSL (or a functional part, derivative and/or analogue thereof) is administered to milk because BSSL is endogenously present in animals. Although BSSL is naturally present in mother's milk, it is preferred to increase the amount of BSSL in order to increase protection against infection. Moreover, the BSSL isoforms present in mother's milk vary between individuals. It is therefore preferred to select at least one BSSL isoform that is particularly effective in counteracting DC-SIGN interactions of pathogens. Milk of individuals who do not produce such effective isoform is preferably enriched with said effective isoform. Moreover, artificial BSSL isoforms with improved characteristics are preferred. In one embodiment at least one preferred isoform of BSSL of the invention, which isoform is particularly well capable of influencing interaction between DC-SIGN and other DC-SIGN ligands, is used in order to more efficiently counteract and/or prevent infection. Non-limiting examples of such preferred isoforms are a human BSSL isoform of between 90 and 115 kDa, preferably between 95 and 110 kDa, most preferably about 102 kDa and a human BSSL isoform having between 13-16, preferably about 14, proline-rich 11-amino acid repeat units in its carboxyl-terminal region. A preferred BSSL isoform according to the invention is preferably added to milk of an individual who does not express said preferred isoform. However, one embodiment comprises enriching milk of individuals who naturally produce said preferred isoform, since increasing the amount of BSSL is beneficial for more efficiently counteracting and/or preventing infection.

A method according to the invention, preferably BSSL or a functional part, derivative and/or analogue thereof, is preferably used in order to counteract infection and/or spread of HIV-1, HIV-2, SIV, hepatitis C, Ebola, cytomegalovirus, Dengue virus, human herpes virus 8, Mycobacterium, Helicobacter pylori, Leishmania, Candida albicans, a gp120 protein, a HCV envelope glycoprotein, a Lewis X sugar motif, a Lewis Y sugar motif, ICAM 2, ICAM 3, β2-integrin Mac-1 (CD11b/CD18) and/or CEACAM1. Most preferably, infection and/or spread of HIV is at least in part prevented.

A food product that has been provided with an isolated, synthetic or recombinant compound comprising at least one Lewis X sugar epitope, or a functional part, derivative and/or analogue thereof, and a non-saccharide moiety is also herewith provided. Said food product preferably comprises milk, more preferably human milk.

It is possible to perform a method according to the invention in vitro as well as in vivo. In vivo applications for instance comprise prophylaxis and/or treatment of pathogenic infections, as described. In vitro applications for instance comprise biomedical research. Routes of infection are for instance investigated and/or modified using a cell line comprising DC-SIGN. Since DC-SIGN is naturally present on dendritic cells, a method according to the invention is preferably applied wherein said DC-SIGN receptor is present on a dendritic cell. However, other applications are possible, such as a use of DC-SIGN in genetically modified cells.

A compound according to the invention is suitable for use as a prophylactic agent against infection by pathogens which are capable of binding DC-SIGN. If an individual is already infected, a compound of the invention is suitable as medicament, in order to at least partly counteract spread of a pathogen and/or subsequent infections. Further provided is therefore a compound comprising at least one Lewis X sugar epitope, or a functional part, derivative and/or analogue thereof, and a non-saccharide moiety for use as a medicament and/or prophylactic agent. One preferred embodiment provides BSSL, or a functional part, derivative and/or analogue thereof, for use as a medicament and/or prophylactic agent. A compound according to the invention is preferably used for preparing a medicament and/or prophylactic agent against a disorder related to infection by a micro organism capable of binding DC-SIGN. Said disorder is preferably related to an HIV-1 infection, an HIV-2 infection, an SIV infection, a hepatitis C infection, an Ebola infection, a cytomegalovirus infection, a Dengue virus infection, a human herpes virus 8 infection, a Mycobacterium infection, a Helicobacter pylori infection, a Leishmania infection, and/or a Candida albicans infection, most preferably to an HIV infection. The invention thus provides a use of a compound comprising at least one Lewis X sugar epitope, or a functional part, derivative and/or analogue thereof, and a non-saccharide moiety, for the preparation of a medicament for at least in part preventing and/or treating a disorder related to infection by a micro organism capable of binding a DC-SIGN receptor. Said compound preferably comprises BSSL or a functional part, derivative and/or analogue thereof. Most preferably, said compound comprises a human BSSL isoform of between 90 and 115 kDa, preferably between 95 and 110 kDa, most preferably about 102 kDa and/or a human BSSL isoform having between 13-16, preferably about 14, proline-rich 11-amino acid repeat units in its carboxyl-terminal region, or a functional part, derivative and/or analogue of said BSSL isoform. In one embodiment said medicament and/or prophylactic agent is capable of at least in part counteracting mucosal absorption and/or mucosal transmission of a micro organism capable of binding a DC-SIGN receptor.

It is also possible to use a compound which is capable of increasing the amount and/or activity of a compound according to the invention. Such compound is capable of indirectly influencing interaction between DC-SIGN and a DC-SIGN ligand. Preferably, a compound capable of increasing the amount and/or activity of endogenously present BSSL is used in order to at least in part counteract infection and/or spread of a micro organism. A compound capable of increasing the amount and/or activity of a compound comprising at least one Lewis X sugar epitope, or a functional part, derivative and/or analogue thereof, and a non-saccharide moiety in an individual for use as a medicament and/or prophylactic agent is therefore also provided by the present invention.

Said compound capable of increasing the amount and/or activity of a compound according to the invention is preferably used for preparing a medicament and/or prophylactic agent against a disorder related to infection by a micro organism capable of binding DC-SIGN. Said disorder is preferably related to an HIV-1 infection, an HIV-2 infection, an SIV infection, a hepatitis C infection, an Ebola infection, a cytomegalovirus infection, a Dengue virus infection, a human herpes virus 8 infection, a Mycobacterium infection, a Helicobacter pylori infection, a Leishmania infection, and/or a Candida albicans infection, most preferably to an HIV infection. The invention therefore provides a use of a compound capable of increasing the amount and/or activity of a compound comprising at least one Lewis X sugar epitope, or a functional part, derivative and/or analogue thereof, and a non-saccharide moiety, for the preparation of a medicament for at least in part preventing and/or treating a disorder related to infection by a micro organism capable of binding a DC-SIGN receptor. Said compound is preferably capable of increasing the amount and/or activity of BSSL. In one embodiment said medicament and/or prophylactic agent is capable of at least in part counteracting mucosal absorption and/or mucosal transmission of a micro organism capable of binding a DC-SIGN receptor.

The invention further provides a composition comprising a compound comprising at least one Lewis X sugar epitope, or a functional part, derivative and/or analogue thereof, and a non-saccharide moiety. Said composition preferably comprises polyacrylamide-Lewis X, bovine serum albumin-Lewis X and/or BSSL, or a functional part, derivative and/or analogue thereof. Most preferably, said composition comprises a human BSSL isoform of between 90 and 115 kDa, preferably between 95 and 110 kDa, most preferably about 102 kDa, and/or a human BSSL isoform having between 13-16, preferably about 14, proline-rich 11-amino acid repeat units in its carboxyl-terminal region, or a functional part, derivative and/or analogue of said BSSL isoform. As explained above, a composition according to the invention is particularly suitable for influencing interaction between DC-SIGN and a DC-SIGN ligand. In one preferred embodiment said composition comprises a pharmaceutical composition. Said composition preferably comprises a pharmaceutically acceptable dose of a compound according to the invention and a pharmaceutically acceptable carrier, diluent and/or excipient.

Dendritic cells, which express DC-SIGN, are involved in an animal's immune response. Now that the invention has provided means and methods for influencing interaction between DC-SIGN and DC-SIGN ligands, interaction between dendritic cells and compounds capable of binding DC-SIGN (such as for instance pathogens) is influenced. A compound according to the present invention thus provides for immunomodulatory activity. Further provided is therefore a method for regulating an immune response in an individual, comprising influencing interaction between a DC-SIGN receptor of said individual and a DC-SIGN ligand with a method according to the invention.

For instance, glycosylation driven binding of both β2-integrin Mac-1 and CEACAM1 to DC-SIGN is required for interaction between neutrophils and dendritic cells which normally leads to the modulation of T cell responses by neutrophils. Cross-talk between neutrophils induces dendritic cells to activate T cell proliferation and to instruct Th1 polarisation. Counteracting interaction between DC-SIGN and β2-integrin Mac-1 and/or CEACAM1 with a method according to the invention provides the possibility to dampen specific T cell responses in cases of over immune activation or where Th2 responses are preferentially required over Th1 responses. An immune response is furthermore regulated by influencing interaction between DC-SIGN and other ligands, preferably a Lewis X sugar motif, a Lewis Y sugar motif, ICAM 2 and/or ICAM 3. One embodiment therefore provides a method according to the invention wherein said DC-SIGN ligand comprises a Lewis X sugar motif, a Lewis Y sugar motif, ICAM 2, ICAM 3, β2-integrin Mac-1 (CD11b/CD18) and/or CEACAM1.

According to the invention, various BSSL isoforms are particularly suitable for influencing interaction between DC-SIGN and DC-SIGN ligands. These isoforms are therefore preferably used for influencing interaction between DC-SIGN and DC-SIGN ligands. Non-limiting examples of such preferred BSSL isoforms are a human BSSL isoform of between 90 and 115 kDa, preferably between 95 and 110 kDa, most preferably about 102 kDa and/or a human BSSL isoform having between 13-16, preferably about 14, proline-rich 11-amino acid repeat units in its carboxyl-terminal region. Some individuals naturally produce at least one of said preferred BSSL isoforms, while other individuals produce other BSSL isoforms. Individuals producing a preferred BSSL isoform according to the present invention are at a lower risk of being infected by a micro organism capable of binding DC-SIGN as compared to other individuals. Moreover, individuals producing a preferred BSSL isoform according to the present invention are at a lower risk of a fast progression of a disease related to a micro organism capable of binding DC-SIGN. One aspect of the invention therefore provides a method for determining whether an individual is at a low risk of being infected by a micro organism capable of binding a DC-SIGN receptor, comprising determining whether said individual comprises a bile salt stimulated lipase (BSSL) isoform having between 13-16, preferably about 14, proline-rich 11-amino acid repeat units in its carboxyl-terminal region and/or a bile salt stimulated lipase (BSSL) isoform of between 90 and 115 kDa, preferably between 95 and 110 kDa, most preferably about 102 kDa. Another aspect of the invention provides a method for determining whether an individual infected by a micro organism capable of binding DC-SIGN is at a low risk of fast progression of a disease related to said micro organism, comprising determining whether said individual comprises a bile salt stimulated lipase (BSSL) isoform having between 13-16, preferably about 14, proline-rich 11-amino acid repeat units in its carboxyl-terminal region and/or a bile salt stimulated lipase (BSSL) isoform of between 90 and 115 kDa, preferably between 95 and 110 kDa, most preferably about 102 kDa.

Moreover, individuals producing a preferred BSSL isoform according to the present invention, who are infected with a micro organism capable of binding DC-SIGN, are at a lower risk of transmitting said micro organism or an antigen of a micro organism which antigen is capable of binding DC-SIGN to a second individual. Particularly, lactating female individuals producing a preferred BSSL isoform according to the present invention, who are infected with a micro organism capable of binding DC-SIGN, are at a lower risk of transmitting said micro organism to their newborn via their milk. Without being bound to theory, it is believed that this effect is achieved because a preferred BSSL isoform present in their milk is better capable of binding DC-SIGN receptors of dendritic cells of their newborn as compared to different BSSL variants present in the milk of other individuals. As a result, pathogens present in milk comprising a preferred BSSL isoform according to the invention are less capable of binding dendritic cells of newborns as compared to pathogens present in milk lacking said preferred isoforms. Hence, if a lactating individual such as a woman or a female non-human animal is known to be infected by a micro organism capable of binding DC-SIGN, such as for instance HIV, it is preferably investigated what BSSL isoforms are produced by said individual. If said individual appears to produce a preferred BSSL isoform according to the invention, she is at a lower risk of transmitting said micro organism to a second individual. Further provided is therefore a method for determining whether a first individual is at a low risk of transmitting a micro organism capable of binding a DC-SIGN receptor to a second individual, comprising determining whether said first individual comprises a bile salt stimulated lipase (BSSL) isoform having between 13-16, preferably about 14, proline-rich 11-amino acid repeat units in its carboxyl-terminal region and/or a bile salt stimulated lipase (BSSL) isoform of between 90 and 115 kDa, preferably between 95 and 110 kDa, most preferably about 102 kDa. Said second individual preferably comprises an individual who receives milk from said first individual.

A preferred BSSL isoform according to the invention is furthermore particularly suitable for at least in part counteracting spread of a pathogen capable of binding DC-SIGN in an individual who is infected by said pathogen. Individuals naturally producing such isoform are at a lower risk of a progressing disease course related to spread of said pathogen. Hence, an infected individual's risk of a progressing disease course is determined by determining whether said individual produces a preferred BSSL isoform according to the present invention. The invention therefore provides a method for determining whether an individual which has been infected by a micro organism capable of binding a DC-SIGN receptor is at a low risk of a progressing disease course, comprising determining whether said individual comprises a BSSL isoform having between 13-16, preferably about 14, proline-rich 11-amino acid repeat units in its carboxyl-terminal region and/or a BSSL isoform of between 90 and 115 kDa, preferably between 95 and 110 kDa, most preferably about 102 kDa.

Now that the invention has provided a compound capable of specifically binding a DC-SIGN receptor, which receptor is present on dendritic cells, it has become possible to specifically target a compound of interest to dendritic cells using a compound according to the invention. A compound of interest is preferably provided with, preferably coupled to, a compound according to the invention. The resulting construct will be specifically targeted to dendritic cells. Further provided is therefore a method for producing a compound which is capable of being specifically targeted to a dendritic cell, comprising providing said compound with at least one Lewis X sugar epitope, or a functional part, derivative and/or analogue thereof, and a non-saccharide moiety. Said compound of interest is preferably provided with polyacrylamide-Lewis X, bovine serum albumin-Lewis X and/or BSSL, or a functional part, derivative and/or analogue thereof. As explained before, BSSL is preferred since this compound is endogenously present in animals. Most preferably, a compound of interest is provided with a human BSSL isoform having between 13-16, preferably about 14, proline-rich 11-amino acid repeat units in its carboxyl-terminal region and/or a human BSSL isoform of between 90 and 115 kDa, preferably between 95 and 110 kDa, most preferably about 102 kDa, since these isoforms are particularly suitable for influencing interaction between DC-SIGN and DC-SIGN ligands.

Said compound of interest preferably comprises a toxic agent, a micro organism, a virus-like particle and/or a pathogen antigen. A toxic agent provided with a compound of the invention capable of specifically binding DC-SIGN is particularly suitable for targeting and eliminating infected dendritic cells. A micro organism, virus-like particle and/or pathogen antigen provided with a compound of the invention capable of specifically binding DC-SIGN is particularly suitable for targeting dendritic cells. This way, specific immune reactions are induced and/or enhanced. A micro organism, virus-like particle and/or pathogen antigen provided with a compound of the invention is therefore particularly suitable for vaccination purposes.

The invention is further illustrated by the following examples. The examples do not limit the scope of the invention in any way.

### EXAMPLES

### Example 1

### Results

The effect of human milk on direct HIV-1 infection of CD4+ T-lymphocytes and DC-SIGN mediated HIV-1 transfer. We investigated the effect of human milk on direct infection of CD4+ T lymphocytes by incubating HIV-1 in the presence of several dilutions of human milk from an HIV-1 negative donor taken 6 months into lactation. The 2 fold dilution of human milk demonstrated a significant degree of viral inhibition (>90%, p<0.05) in comparison to the PBS control (FIG. 1A). We also identify an enhancement to infection at the 1/10 dilution. Pre-incubation of the same human milk sample (1/2) with HIV-1 before addition to CD4⁺ T-lymphocytes (FIG. 1B) showed no inhibitory effect in comparison to the PBS control, indicating that the inhibition observed in FIG. 1A is not due to a direct virucidal effect of the milk and that the effect is likely conferred on the CD4⁺ lymphocyte. The experiments were repeated with human milk from two other mothers with equivalent results obtained (data not shown).

DC-SIGN expressing cells enhance infection of CD4⁺ T-lymphocytes. To study the effect of human milk on DC-SIGN mediated transfer of HIV-1 we utilized the Raji cell-line expressing the DC-SIGN receptor (Raji-DC-SIGN) (Wu et al, 2004). The same human milk sample as used in the direct infection assay or PBS spiked with HIV-1 primary isolates was incubated with the Raji-DC-SIGN cells for 30 minutes or 2 hours, after which the cells were washed and incubated with activated CD4⁺ T-lymphocytes and the culture monitored for viral replication. Raji-DC-SIGN cells pre-incubated with PBS spiked with HIV-1 showed efficient transfer to CD4⁺ T-lymphocytes (FIG. 2A, squares). The Raji cell line not expressing DC-SIGN showed no viral transfer demonstrating our effect is DC-SIGN dependent (data not shown). Surprisingly, pre-incubation of Raji-DC-SIGN cells with HIV-1 spiked human milk significantly reduced or blocked transfer of HIV-1 depending on the incubation time (FIG. 2A, diamonds). Through testing cell viabilities we demonstrated that the observed inhibition was not due to induced cell death of Raji-DC-SIGN by human milk (data not show). The transfer experiment revealed that human milk blocks the transfer of HIV-1 by Raji-DC-SIGN cells irrespective of viral co-receptor using phenotype. We next performed limiting dilutions of the same human milk sample and identified that R5HIV-1 variants (viruses that utilize the CCR5 co-receptor for viral entry) and X4 HIV-1 variants (viruses utilizing the CXCR4 co-receptor) were completely inhibited at a 1/128 but not at a 1/512 dilution (FIG. 2B). Similar results were observed with the same assays using human milk from two other HIV-1 negative donors (data not shown). Human milk compound(s) bind to DC-SIGN thereby preventing transfer of HIV-1 to CD4⁺ T lymphocytes. To determine whether the inhibitory effect of human milk on Raji-DC-SIGN mediated viral transfer was caused by interaction of human milk with HIV-1 or DC-SIGN we conducted pre-incubation experiments of either Raji-DC-SIGN cells or HIV-1 with human milk. To test for binding of the inhibitory factor to Raji-DC-SIGN cells we pre-incubated the cells with either human milk or PBS before washing and then adding virus and subsequently CD4⁺ T-lymphocytes. Alternatively, to test binding of components in human milk to the virus we incubated a high titer virus stock with either PBS or human milk after which the Raji-DC-SIGN cells were added and thereby diluting the milk to a non-inhibitory concentration (1/667), after which CD4⁺ T-lymphocytes were added. Pre-incubation of virus with human milk demonstrated a slight reduction in viral transfer compared to the PBS control (FIG. 3A), likely reflecting residual inhibitory effects of the human milk. In contrast, pre-incubation of the Raji-DC-SIGN cells with human milk provided a highly significant reduction in viral transfer compared to the PBS control (p<0.01) (FIG. 3B). To test whether the observed inhibition was due to down-modulation of DC-SIGN expression we investigated the surface expression of DC-SIGN in the presence of human milk. We demonstrate that with two DC-SIGN specific monoclonal antibodies, AZN-D2 and anti-stalk #4, cell surface expression was not altered, whereas the binding of AZN-D1 was reduced when DC-SIGN cells were pre-incubated with human milk (FIG. 3C). These results suggest that the inhibitory effect is mediated via the binding of factor(s) in human milk to the DC-SIGN molecule and preventing its interaction with HIV-1 opposed to the down-modulation of the DC-SIGN molecule at the cell surface.

To show direct binding of human milk compound(s) to DC-SIGN we introduced two previously described assays (Geijtenbeek et al, 2000 a; Geijtenbeek et al, 2002), the gp120 bead adhesion assay and the DC-SIGN-Fc binding ELISA. In the gp120 bead adhesion assay, the effect of human milk on the binding of gp120 coated fluorescent beads to cellular DC-SIGN was studied. The binding of the gp120 beads to both Raji-DC-SIGN and iDC was inhibited by human milk in comparison to the control (p<0.01) (FIGS. 4A & 4B, respectively). To demonstrate DC-SIGN specific binding the cells were pre-incubated with a DC-SIGN specific antibody (AZN-D1), the DC-SIGN binding sugar mannan, and the Ca²⁺ chelater EGTA. These agents were found to block binding of gp120 beads to the DC-SIGN expressing cells to the same extent as human milk (FIGS. 4A & 4B). To confirm the direct interaction of DC-SIGN with human milk we performed a DC-SIGN-Fc binding ELISA where we demonstrate that DC-SIGN-Fc binding to human milk was specific since pre-incubation of DC-SIGN-Fc with AZN-D1 or EGTA completely abrogated binding (p<0.01) (FIG. 4C).

The liver and lymph node specific homologue of DC-SIGN, L-SIGN, is also capable of interacting with HIV-1 and enhancing viral infectivity. To investigate the effect of human milk on the interaction of b-SIGN and gp120 we incubated Raji cells expressing the L-SIGN molecule (Raji-L-SIGN) with human milk in the gp120 bead adhesion assay and as a control the cells were pre-incubated with AZN-D1, AZN-D2, a L-SIGN specific antibody, mannan and EGTA. The result demonstrates that there is no inhibition of human milk on the interaction of gp120 with L-SIGN (FIG. 4D), indicating a specificity of the human milk compound for DC-SIGN.

Human milk inhibits both iDC and mDC dependent transfer of HIV-1. To study the biological relevance of the inhibitory properties of human milk we utilized a previously described single cycle HIV-1 transmission assay (Groot et al, 2005) using both iDCs and mDCs. Either iDCs or mDCs (matured by poly I:C) were incubated with several dilutions of human milk before addition of HIV-1 and target cells. After 24 hours luciferase activity was measured representing transmission of HIV-1 to the LuSIV cells. Human milk was found to inhibit HIV-1 mediated transfer by both iDCs and mDCs (FIG. 5A), in a dose dependent manner with the 2 and 5 fold dilutions showing significant inhibition for both cell types (p<0.01). Washing after the pre-incubation of mDCs with human milk (1/2) before addition of HIV-1 also showed a significant reduction (p<0.01) of HIV-1 transfer in comparison to the PBS control (data not shown), indicating that the observed inhibition in FIG. 5A is not due to a direct virucidal effect of the human milk. We also demonstrate that a 1/2 and 1/5 dilution of human milk significantly reduces HIV-1 capture by iDCs (P<0.01) (FIG. 5B). We also tested for DC-SIGN down-modulation through cell surface staining with the different antibodies directed against DC-SIGN (AZN-D1, AZN-D2 and anti-stalk #4) in the presence or absence of human milk. Both the AZN-D2 and anti-stalk #4 show no significant difference in binding before and after human milk exposure, indicating that the DC-SIGN receptor is not down-modulated by the interaction with human milk (FIG. 5C) whilst AZN-D1 shows a reduction in binding as was observed with the Raji-DC-SIGN cells (FIG. 3C).

Major human milk proteins do not bind DC-SIGN nor inhibit HIV-1 transfer to CD4⁺ T-lymphocytes. Since inhibition by human milk is present at relatively high dilutions (FIG. 2B) we hypothesized that one of the major proteins present in human milk may be responsible for the activity. We therefore tested human lactoferrin, bovine β-casein, human lysozyme, human α-lactalbumin and human SLPI which have been shown to posses modulatory effects on HIV-1 replication in vitro. All these compounds were tested in the gp120 bead adhesion assay and the DC-SIGN-Fc binding ELISA (FIG. 6). None of the tested human milk compounds could inhibit gp120 binding to Raji-DC-SIGN or iDCs (FIGS. 6A & B, respectively). Furthermore, DC-SIGN-Fc showed no binding to the selected milk compounds (FIG. 6C). As a control bovine lactoferrin was used which has previously been shown to bind DC-SIGN and prevent viral transfer (58).

Pre-incubation of human milk with an anti-Le^{x} antibody lifted the inhibition of HIV-1 transfer. Since DC-SIGN can bind sugars we hypothesized that one of the abundant sugar motifs in human milk may provide the inhibitory activity. We predicted that Le^{x} could be contributing to the observed inhibition. To test our hypothesis we pre-incubated human milk with an anti-Le^{x} IgM antibody or an IgM isotype control before use in the culture transfer assay. The results demonstrated a dose dependent lifting of the inhibitory effects of human milk on viral transfer after a pre-incubation with the Le^{x} specific antibody but not with the control antibody (FIG. 7A). In contrast, the gp120 bead adhesion assay did not show reduced binding of DC-SIGN with Le^{x} antibody (data not shown). The most likely explanation for this difference is that the low affinity Le^{x} specific antibody was unable to block the high avidity interaction of cellular multimeric DC-SIGN to the unknown human milk component. The DC-SIGN-Fc binding ELISA (FIG. 7B) demonstrated a reduction in DC-SIGN-Fc binding after pre-incubation of the coated human milk with the Le^{x} specific antibody but again not with the control antibody. These results indicate that Le^{x} is critical for the human milk compound that binds to the DC-SIGN molecule.

Not all Le^{x} containing complexes can inhibit HIV-1 interacting with the DC-SIGN receptor. We tested several compounds containing one or more Le^{x} epitopes for inhibitory and DC-SIGN binding activity. In the transfer culture assay, Le^{x} coupled to biotinylated polyacrylamide (PAA-Le^{x}) and Le^{x}-BSA were both able to inhibit DC-SIGN mediated HIV-1 transfer (FIG. 8A). On the contrary both LNFP III, a Le^{x} containing oligosaccharide present in human milk, and the Le^{x} trisaccharide itself were not able to prevent HIV-1 transfer (FIG. 8A), even though LNFP III has been shown previously to bind DC-SIGN (Guo et al, 2004). The compounds were also tested in the gp120 bead adhesion assay, but none of the tested compounds were able to block the interaction between DC-SIGN and gp120 (data not shown). Most likely, PAA-Le^{x} and Le^{x}-BSA are able to block the trimeric gp120 interaction while binding of DC-SIGN to monomeric gp120 expressed on the fluorescent beads is still possible. Due to the inability to coat saccharides onto plates in the DC-SIGN-Fc binding ELISA we only tested Le^{x}-BSA and the BSA control. Le^{x}-BSA indeed demonstrates binding to DC-SIGN-Fc, whereas BSA showed no binding, indicating that the DC-SIGN-Fc binding is Le^{x} specific (FIG. 8B).

### Methods

Cells. The Raji control cell line and the cell lines expressing either DC-SIGN (Raji-DC-SIGN) (Geijtenbeek et al, 2000 b) or L-SIGN (Raji-L-SIGN) were cultured in RPMI containing 10% fetal calf serum (FCS), penicillin (100 units/ml), streptomycin (100 units/ml), and neomycin (2 mg/ml) for the Raji-DC-SIGN and Raji-L-SIGN cells. Peripheral Blood Mononuclear Cells (PBMCs) were isolated from three buffy coats by standard Ficol-Hypaque density centrifugation, pooled and frozen in multiple vials. After thawing PBMCs were activated with phytohemagglutinin (3 μg/ml) and cultured in RPMI medium containing 10% FCS, penicillin (100 units/ml) and streptomycin (100 units/ml) with recombinant interleukin-2 (100 units/ml). On day 3 the cells underwent CD4+ enrichment by incubation with CD8 immunomagnetic beads (Dynal) and negatively selected according to the manufacturers instructions and cultured with IL-2 (100 units/ml). Dendritic cells for the single-cycle transmission assay were generated from fresh PBMCs, cells were layered on a standard Percoll gradient (Pharmacia). The light fraction with predominantly monocytes was collected, washed, and seeded in 24-well or 6-well culture plates at a density of 5×10⁵ cells or 2.5×10⁶ per well, respectively. After 60 min at 37° C. the adherent cells were cultured to obtain immature DCs in Iscove's modified Dulbecco's medium (IMDM) with gentamicin (86 μg/ml) and 10% fetal clone serum (Hyclone) and supplemented with granulocyte-macrophage colony-stimulating factor (GM-CSF) (500 U/ml) and Interleukin-4 (IL-4) (250 U/ml). Culture medium was refreshed on day 3 with cell maturation induced at day 6 by culturing with poly(I:C) (20 μg/ml) (Sigma-Aldrich). After two days mature CD14⁻ CD1b⁺ CD83⁺ DCs were obtained, washed and utilized. The LuSIV cells with an integrated long terminal repeat-luciferase reporter construct used to measure the transmission in the single cycle replication assay have previously been described (Roos et al, 2000). Cells were cultured in RPMI 1640 with 10% FCS, 2 mM sodium pyruvate, 10 mM HEPES, 2 mM L-glutamine and penicillin (100 U/ml), streptomycin (100 μg/ml) and hygromycin B (300 μg/ml). The day prior to the single-cycle-replication experiment the cells were plated 1:3 in fresh medium without hygromycin B.

Viruses. Replication competent HIV-1 stocks were generated by the passage of viruses through CD4⁺ lymphocytes with Tissue Culture Infectious Dose (TCID₅₀/ml) determined by limiting dilution on CD4⁺ enriched lymphocytes (Pollakis et al, 2001). Subtype B molecular cloned viruses JR-CSF (R5), LAI (X4), SF-162 (R5) and subtype B primary isolates NSI-18 (R5) and SI-19 (X4) were used in the experiments.

Human milk fractions and commercial milk products. Human milk was collected from three mothers. Since one-off samplings were obtained from the mothers, internal review board approval was not required. The milk was centrifuged at 400×g for 10 minutes followed by centrifugation at 530×g for 10 minutes with pipette removal of the lipid layer and cells after each centrifugation step. The human milk samples were sterilized by sequential filtration through 0.45 μm and 0.2 μm syringe filters (Schleicher & Schuell) and stored at −80° C. Human lactoferrin (Sigma-Aldrich), bovine lactoferrin (Sigma-Aldrich), human α-lactalbumin (Sigma), bovine β casein (NIZO Food Research), human lysozyme (Sigma), LNFP III (Calbiochem), SLPI (Sigma), Lewis X trisaccharide (Calbiochem), Lewis X-BSA, 14 atom spacer (Calbiochem), Le^{x} coupled to biotinylated polyacrylamide (PAA-Le^{x}) (Syntesome), anti-human Le^{x} (mouse) IgM (Calbiochem) and anti-human ARA-LAM (mouse) IgM were used. All compounds were utilized at physiological relevant concentrations where known by dilution in phosphate buffered saline (PBS) containing 10% FCS.

Direct HIV-1 infection assay. Enriched CD4⁺ lymphocytes were plated in 96 well plates at 1×10⁵ cells/well in the presence of RPMI media containing 10% FCS and IL-2 (100 units/ml) with penicillin (100 units/ml) and streptomycin (100 units/ml). Cells were incubated for 2 hours with human milk diluted in PBS containing 10% FCS and spiked with 3.7log TCID₅₀/ml of HIV-1. After 2 hours the cells were washed and fresh RPMI containing 10% FCS, penicillin (100 units/ml), streptomycin (100 units/ml) and IL-2 (100 units/ml) was added. Alternatively, after a 2 hour incubation of human milk with HIV-1, the spiked milk was diluted with PBS containing 10% FCS before addition to CD4⁺ T-lymphocytes. On day 7 of culture viral p24 levels in the culture supernatant was determined by a standard ELISA.

DC-SIGN mediated HIV-1 transfer assay. The Raji and Raji-DC-SIGN cells were plated at a concentration of 2×10⁴ cells/well in a 96 well format. Dilutions of human milk or human milk compounds were made in PBS containing 10% FCS and spiked with 3.7log TCID₅₀/ml of the appropriate virus before addition to the Raji-DC-SIGN cells. As controls PBS containing 10% FCS was spiked with the same TCID₅₀/ml of the corresponding virus before addition to Raji or Raji-DC-SIGN cells. After incubation the culture was washed with PBS before addition of CD4⁺ enriched T-lymphocytes at a concentration of 1×10⁵ cells/well with p24 determined on day 7. DC-SIGN specific antibody binding after exposure to human milk. Human milk was incubated with 50×10³ Raji-DC-SIGN or iDCs for 15 minutes at 37° C. after which the cells were washed with TSM (20 mM Tris, 150 mM NaCl, 1 mM CaCl₂, 2 mM MgCl₂) and the cells were incubated at 4° C. for 45 minutes with 5 μg/ml of the specific DC-SIGN antibody, AZN-D1 (Geijtenbeek et al, 2000 a), AZN-D2 (Halary, 2002) or anti-stalk #4. Subsequently, the cells were washed and incubated with goat anti-mouse FITC, for 45 minutes at 4° C. The cells were washed and resuspended in 100 μl TSM containing 0.5% bovine serum albumin, (BSA Fraction V, Fatty Acid-Free) (Calbiochem) and the adhesion was measured by flow cytometry (BD Biosciences).

DC-SIGN Binding assay. Pre-incubation of HIV-1 with human milk: a 1/4 dilution of human milk or PBS containing 10% FCS was incubated with a high virus titer (5.6 log TCID₅₀/ml) of SF-162 for 1 hour at 37° C. before diluting the mixture to 1/667 by addition of 0.27×10⁶ Raji-DC-SIGN cells in a 24-wells format. The cells were incubated for 2 hours at 37° C. before washing with PBS and addition CD4⁺ T lymphocytes at a concentration of 1×10⁶ cells/ml. Virus replication was monitored by measuring p24 production on day 15 by standard ELISA. Pre-incubation of Raji-DC-SIGN cells with human milk: a dilution of the human milk (1/4) or PBS containing 10% FCS was incubated with 0.27×10⁶ Raji-DC-SIGN cells in a 24-well format for 1 hour at 37° C. before washing with PBS and the addition of SF-162 (2.8 log TCID₅₀/ml). The cells were incubated for 2 hours before washing and adding 1×10⁶ cells/well of CD4⁺ enriched lymphocytes. Virus replication was monitored by measuring p24 production on day 15 by standard ELISA.

gp120 bead adhesion assay. Beads were prepared as previously described (Geijtenbeek et al, 2000 a), in short, streptavidin was covalently coupled to Carboxylate-modified TransFluoSpheres (488/645 nm, 1.0 μm; molecular probes). The steptavidin-beads were incubated with biotinylated F(ab′)₂ fragment goat-anti-human IgG (6 μg/ml; Jackson Immunoresearch) and subsequently incubated overnight with gp120-Fc chimera. Fifty thousand cells, Raji-DC-SIGN cells, immature DCs or Raji-L-SIGN cells, were pre-incubated with human milk or milk compounds, AZN-D1 (Geijtenbeek et al, 2000 a), AZN-D2 (Halary et al, 2002), EGTA or mannan for 30 minutes at RT. The ligand-coated beads (20 beads/cell) were added to the pre-incubated cells and incubated for 30 minutes at 37° C. after which the cells were washed with TSM containing 0.5% BSA. After washing, the cells were resuspended in 100 μl TSM-BSA buffer and the adhesion was measured by flow cytometry (BD Biosciences).

DC-SIGN-Fc binding ELISA. The DC-SIGN-Fc chimera contained the extracellular portion of DC-SIGN (amino acids 64 to 404) fused at the C-terminus to a human immunoglobulin (Ig) G1 Fc fragment and has been previously described (Geijtenbeek et al, 2002). Human milk or human milk compounds were diluted in 0.2M NaHCO₃, coated on ELISA plates (maxisorb plate; Nunc) and incubated overnight at 4° C. or 2 hours at 37° C. This was followed by blocking with TSM containing 1% BSA for 30 minutes at 37° C. before addition of soluble DC-SIGN-Fc (5 μg/ml) for 2 hours at RT, the binding was determined by incubation of a peroxidase labeled anti-IgG1 antibody for 30 minutes at RT. DC-SIGN-Fc binding specificity was determined by pre-incubation of the DC-SIGN-FC with either 50 μg/ml DC-SIGN specific mouse antibody AZN-D1 (Geijtenbeek et al, 2000 a) or 10 mM EGTA. Single-cycle-replication transmission assay. The assay was performed as previously described (Groot et al, 2005). In short mDCs and iDCs were incubated in a 96-well-plate (35−50×10³ DC/well) with human milk for 30 minutes at 37° C. before addition of the virus (5 ng p24/well), which was incubated for 2 hours at 37° C. The DCs were washed twice with PBS before addition of 50×10³ LuSIV cells. After 24 hours, LuSIV cells were harvested and resuspended in 50 μl lysis buffer (25 mM Tris-Cl 7.8, 2 mM DTT, 2 mM CDTA, 10% glycerol, 1% Triton X100). The cells were then incubated for 45 minutes at room temperature while shaking, followed by 10 minutes centrifugation at 3200×g. The supernatant was transferred to a white-solid 96 well plate (Corning Costar), and 150 μl of lucibuffer (100 μg/ml BSA, 6.6 mM ATP, 15 mM MgSO₄, 25 mM glycylglycine) was added. 100 μl of DE(−)Luciferin (Roche Diagnostics GmbH) was injected per well (0.28 mg/ml lucibuffer excluding ATP). 50×10³ LuSIV cells grown without DC or HIV-1 were used to obtain the background luciferase value.

Capture assay. To measure the capture of HIV-1 by iDCs the cells were incubated in a 96-well-plate (50×10³ DC/well) with human milk for 30 minutes at 37° C. before addition of the virus (5 ng p24/well), which was incubated for 2 hours at 37° C. The iDCs were washed twice with PBS before the p24 concentration was determined by standard ELISA.

### Example 2

### Results

Biochemical analysis of the DC-SIGN binding compound in human milk. In order to determine whether the inhibitory activity of human milk is protein associated or not we isolated the protein fraction from milk S3 using a C18 column and tested this along with the untreated sample in the gp120 bead adhesion assay utilizing immature DCs (iDCs). The human milk protein fraction showed inhibition of gp120 binding although slightly less than that observed with the untreated milk, indicating that the inhibitory activity was maintained within the protein fraction (FIG. 9A). The AZN-D1 Ab, mannan and EGTA controls all demonstrated inhibition indicating that the observation is DC-SIGN mediated. To further confirm that the inhibitory compound is a protein we trypsin treated milk S3 and tested this in the DC-SIGN-Fc binding ELISA (FIG. 9B). Human milk incubated with medium showed DC-SIGN specific binding whereas the trypsin treated milk showed a significant reduction in binding to DC-SIGN (P<0.01), although not all activity was lost. This result was confirmed in the DC-SIGN-mediated HIV-1 transfer assay which demonstrated a reduced inhibition of HIV-1 infection with the trypsin treated human milk in comparison to the untreated sample (data not shown). These results reiterate that the inhibitory compound in human milk is either protein or protein associated.

We next tested whether heating of the human milk S3 could alleviate DC-SIGN binding or HIV-1 inhibitory activity. Heating the milk to 99° C. induced no loss of DC-SIGN-Fc binding in either the ELISA assay (FIG. 9C) or in the gp120 bead adhesion assay with either Raji-DC-SIGN cells or iDC (data not shown). Heating the milk sample also did not alleviate inhibition in the HIV-1 viral transfer assay (data not shown). These results taken into consideration with our results of Example 1 indicate that the protein does not loose function when the original tertiary structure is lost and suggests that the attached Le^{x} sugars are the active component.

The >100 kDa fraction contains the active compound in human milk. To gain a better understanding of the protein size responsible for the inhibitory activity we performed size fractionation of human milk to determine in which fraction(s) the active component is present. S3 was fractionated with the derived fractions tested in the DC-SIGN-Fc binding ELISA (FIG. 10A). Specific binding to DC-SIGN was only observed in the >100 kDa fraction. The non-specific binding observed for the 30-100 kDa fraction could be explained by the binding of the anti-human-Fc secondary Ab to the milk immunoglobulins. The results of the gp120 bead adhesion to iDC and Raji-DC-SIGN (data not shown) as well as the Raji-DC-SIGN culture assay (FIG. 10B) confirm that only the >100 kDa fraction is able to inhibit gp120 binding to DC-SIGN and prevent viral transfer (P<0.01).

Variation in binding activity between human milk samples from three different mothers. To determine whether variability in the DC-SIGN binding capacity of human milk exists between mothers we tested milk samples from three individuals (S1, S2 and S3). The three samples were tested in the DC-SIGN-Fc ELISA (FIG. 11A) and the DC-SIGN transfer culture assay (FIG. 11B). In the DC-SIGN-Fc ELISA both S1 and S2 demonstrate low to no binding differences compared to the relevant AZN-D1 and EGTA controls, whereas S3 shows increased binding (P<0.01) in comparison to AZN-D1 and EGTA. The DC-SIGN-FC ELISA results were confirmed by the DC-SIGN transfer culture assay (FIG. 11B), which showed a significant (P<0.02) loss of inhibition at a dilution of 1:256 for the S1 and S2 in comparison to S3, which still showed significant inhibition (P<0.02) of HIV-1 transfer at a dilution of 1:2048. These results demonstrate that the inhibitory activity is significantly different between mothers.

Identification of BSSL as an inhibitory glycoprotein. In order to identify the inhibitory compound in human milk we performed western-blot analysis with both Le^{x} Ab and the DC-SIGN-Fc product. Since we had identified differences in DC-SIGN binding with different milk samples we predicted that we should be able to identify alterations in expression of specific protein bands or alterations in glycosylation of specific products. We performed western-blot staining of human milks S1, S2 and S3, which were developed with a LeX specific antibody (FIG. 12A). When comparing the different samples we observe for S3 a band higher than 100 kDa that is not present in S1 or S2 indicating a difference in Le^{x} expression. The Le^{x} staining result also demonstrates that the lower molecular weight proteins can be efficiently glycosylated to contain Le^{x} epitopes and that the levels are similar between all mothers. This observation would suggest that these lower Le^{x} associated molecular weight proteins are not providing for the observed variation in inhibitory activity, which fits with the size fractionation result showing that the responsible protein is >100 kDa and our finding that not all Le^{x} containing compounds can bind DC-SIGN (see Example 1). In the DC-SIGN-Fc stained western-blot (FIG. 12B) S3 shows a band at a similar molecular weight which is not as pronounced for S1 and S2. We also observe that the equivalent band from S1 and S2 runs higher in the gel than the protein from S3. These results correspond to the differences in inhibitory activity observed between the samples. The coomassie stained SDS-PAGE gel (FIG. 12C) also shows a band at the corresponding molecular weight and reconfirms that the bands in the human milk S1 and S2 runs higher than the band of interest in S3. The diffusion of the band indicates that this protein is a glycoprotein in accordance with our hypothesis.

We next extracted the bands from the SDS-PAGE gel of S1, S2 and S3. The peptide mass fingerprint analysis of the selected protein bands identified it as human bile salt-activated lipase (AAA63211) with 20 peptides out of 35 matching (at 30 ppm or below). The sequence coverage was 32% and the Probability based MOWSE score 199 (with protein scores greater than 76 considered significant).

BSSL inhibits DC-SIGN binding and DC-SIGN mediated transfer of HIV-1. To confirm that BSSL serves as an inhibitory compound in human milk we isolated BSSL from S4. The isolated BSSL was tested in the DC-SIGN transfer culture assay (FIG. 13A) and showed significant inhibition at 30 μg/ml and 1.2 μg/ml (P<0.05). These results were confirmed by the DC-SIGN-Fc ELISA (FIG. 13B) which showed binding at concentrations of 30 μg/ml, 3 μg/ml and 0.3 μg/ml BSSL (P<0.01) illustrating that BSSL indeed binds to DC-SIGN and inhibits DC-SIGN mediated transfer of HIV-1 to CD4⁺ T-lymphocytes. In order to show that the BSSL fits with our characterization of the human milk inhibitory factor we performed trypsinization and heat treatment of the purified BSSL and demonstrated that BSSL is heat resistant and trypsin sensitive corresponding to the results obtained with human milk. (data not shown). Interestingly, as with the human milk sample the BSSL binding is not fully alleviated with trypsin treatment suggesting a partial sensitivity. BSSL inhibitory activity is blocked with Le^{x} antibodies. To confirm our hypothesis that Le^{x} is the active component of the glycoprotein BSSL, we pre-incubated human milk with the Le^{x} IgM before addition of DC-SIGN-Fc in the DC-SIGN-Fc binding ELISA. The DC-SIGN binding capacity of BSSL was blocked by pre-incubation with the Le^{x} specific Ab (FIG. 14A), confirming that the Le^{x} expressed by BSSL is the DC-SIGN binding compound. We also tested two mAb's raised against BSSL, N or C-terminus directed, but neither of these Ab's were able to block BSSL from binding DC-SIGN (FIG. 14B). Two isoforms of BSSL from the same human milk sample show a difference in DC-SIGN binding capacity. To further analyze the correlation between the size of BSSL and the DC-SIGN binding capacity we isolated two isoforms, variant in size, from the same mother. The larger isoform (132 kDa) demonstrated a decrease in DC-SIGN binding compared to the smaller form (102 kDa) of BSSL (FIG. 15), demonstrating that differences in BSSL isoforms are related to differences in binding between mothers. Since the two bands are isolated from a single milk sample from the same mother then these different binding patterns are unlikely to be due to variations in glycosylation patterns or sugar modifications.

### Methods

Cells. The Raji control cell line and the cell line expressing DC-SIGN (Raji-DC-SIGN) (Geijtenbeek et al, 2000 b) were cultured in RPMI containing 10% fetal calf serum (FCS), penicillin (100 units/ml), streptomycin (100 units/ml), and neomycin (2 mg/ml) for the Raji-DC-SIGN cells. Peripheral Blood Mononuclear Cells (PBMCs) were isolated from three buffy coats by standard Ficol-Hypaque density centrifugation, pooled and frozen in multiple vials. After thawing PBMCs were activated with phytohemagglutinin (2 μg/ml) and cultured in RPMI medium containing 10% FCS, penicillin (100 units/ml) and streptomycin (100 units/ml) with recombinant interleukin-2 (100 units/ml). On day 3 the cells underwent CD4⁺ enrichment by incubation with CD8 immunomagnetic beads (Dynal) and negatively selected according to the manufacturers instructions and cultured with IL-2 (100 units/ml).

Viruses. Replication competent HIV-1 stocks were generated by the passage of viruses through CD4⁺ lymphocytes with Tissue Culture Infectious Dose (TCID₅₀/ml) determined by limiting dilution on CD4⁺ enriched lymphocytes (Pollakis et al, 2001). Subtype B molecular cloned viruses LAI (X4) was used as the virus in all experiments.

Biochemical analyses of human milk. Human milk was loaded on to a C18 column and the filtrate containing the free human milk saccharides was collected and stored at −20° C. until further use. Human milk was incubated with trypsin-EDTA (1×) (Invitrogen) or RPMI (Invitrogen) for 3 hours while shaking at 37° C., after which the trypsin was inactivated by heating at 95° C. for 10 minutes. The treated human milk samples were stored at −80° C. until used. Human milk was fractionated with the use of microcon centrifugal Filter devices (Millipore), sizes 3,000; 10,000; 30,000 and 100,000 NMWL. First the milk was loaded on the 3,000 NMWL filter device, the remanence was loaded to the next filter, with compensating for lost volume with PBS. The standard manufacturers protocol was followed to obtain the different fractions. Human milk and BSSL. Human milk samples were collected from three mothers (S1, S2 and S3) in Amsterdam, the Netherlands. S1 and S3 were taken 6 months into lactation and sample S2 was taken at an unknown timepoint. Since one-off samplings were obtained from the mothers, internal review board approval was not required. The milk was centrifuged at 400×g for 10 minutes followed by centrifugation at 530×g for 10 minutes with pipette removal of the lipid layer and cells after each centrifugation step. The human milk samples were sterilized by sequential filtration through 0.45 μm and 0.2 μm syringe filters (Schleicher & Schuell) and stored at −80° C. Milk samples were also collected from two additional mothers in Umea, Sweden, from which BSSL was isolated (S4 and S5).

BSSL was isolated from human milk according to the protocol previously described (Blackberg et al, 1981) but in our protocol instead of using an affi-Gel blue sepharos we used a second heparin sepharose purification. Collected fractions were analyzed for BSSL by lipase activity, SDS-PAGE and immunoblotting.

gp120 bead adhesion assay. Beads were prepared as previously described (Geijtenbeek et al, 2000 a), in short, streptavidin was covalently coupled to Carboxylate-modified TransFluoSpheres (488/645 nm, 1.0 μm; molecular probes). The steptavidin-beads were incubated with biotinylated F(ab′)₂ fragment goat-anti-human IgG (6 μg/ml; Jackson Immunoresearch) and subsequently incubated overnight with the gp120-Fc chimera. Fifty thousand cells, Raji-DC-SIGN cells or iDCs were pre-incubated with human milk, AZN-D1 (Geijtenbeek et al, 2000 a), AZN-D2 (Halary et al, 2002), EGTA or mannan for 30 minutes at RT. The ligand-coated beads (20 beads/cell) were added to the pre-incubated cells and incubated for 30 minutes at 37° C. after which the cells were washed with TSM (20 mM Tris, 150 mM NaCl, 1 mM CaCl₂, 2 mM MgCl₂) containing 0.5% BSA. After washing, the cells were resuspended in 100 μl TSM-BSA buffer and the adhesion was measured by flow cytometry (BD Biosciences).

DC-SIGN-Fc binding ELISA. The DC-SIGN-Fc chimera contained the extracellular portion of DC-SIGN (amino acids 64 to 404) fused at the C-terminus to a human immunoglobulin (Ig) G1 Fc fragment which has been previously described (Geijtenbeek et al, 2002). Human milk or BSSL were diluted in 0.2 M NaHCO₃, coated on ELISA plates (maxisorb plate; Nunc) and incubated overnight at 4° C. or 2 hours at 37° C. This was followed by blocking with TSM containing 1% BSA for 30 minutes at 37° C. before addition of soluble DC-SIGN-Fc (5 μg/ml) for 2 hours at RT, the binding was determined by incubation of a peroxidase labeled anti-IgG1 antibody for 30 minutes at RT. DC-SIGN-Fc binding specificity was determined by pre-incubation of the DC-SIGN-Fc with either 50 μg/ml DC-SIGN specific mouse antibody AZN-D1 (Geijtenbeek et al, 2000 a) or 10 mM EGTA.

DC-SIGN mediated HIV-1 transfer assay. The Raji and Raji-DC-SIGN cells were plated at a concentration of 2×10⁴ cells/well in a 96 well format. Dilutions of human milk or BSSL were made in PBS containing 10% FCS and spiked with 3.7log TCID₅₀/ml of the appropriate virus before addition to the Raji-DC-SIGN cells. As controls PBS containing 10% FCS was spiked with the same TCID₅₀/ml of virus before addition to Raji or Raji-DC-SIGN cells. After a two hour incubation the culture was washed with PBS before addition of CD4⁺ enriched T-lymphocytes at a concentration of 1×10⁵ cells/well with p24 values determined on day 7.

Western blots and Coomassie staining. The concentration of the human milk samples was standardized to 30 μg/ml and separated on 8% SDS-PAGE gels (BioRad). The gel was transferred to polyvinylene diflouride membranes (Millipore), and developed with a mouse anti-human Le^{x} Ab, C3D-1 (Santa Cruz) (0.2 μg/ml) and a mix of two different goat anti mouse IgG antibodies (biorad, 0.07 μg/ml and biosource, 1/10,000) or the membrane was stained with DC-SIGN-Fc (600 μg/ml) and a goat anti human IgG (Jackson). Visualization was performed using enhanced chemiluminescence (Amersham Biosciences, Inc.). For the coomassie staining 60 μg of each human milk sample was loaded on an 8% SDS-PAGE. The gel was stained with 50% methanol, 2% acetic acid and 0.25% coomassie after which the gel was destained with 30% methanol and 2% acetic acid, the gel was stored in water with 1% acetic acid at 4° C. for further analyzes.

MALDI protein identification. The protein bands of interest were cut from the gel after staining. For mass spectrometry analysis the gel slices were S-alkylated with iodoacetamide and vacuum dried using a speedvac. The in-gel digestion with trypsin (Roche Molecular Biochemicals, sequencing grade) and extraction of the peptides after the overnight incubation were done according to Shevchenko et al. (Shevchenko et al, 1996). The collected eluates were dried overnight in a speedvac. The peptides were redissolved in 6 μl of a solution containing 1% formic acid and 60% acetonitrile. The peptide solutions were mixed 1:1 (v/v) with a solution containing 52 mM α-cyano-4-hydroxycinnamic acid (Sigma-Aldrich Chemie BV) in 49% ethanol/49% acetonitril/2% TFA and 1 mM Ammoniumacetate. Prior to dissolving, the α-cyano-4-hydroxycinnamic acid was washed briefly with acetone. The mixture was spotted on a target plate and allowed to dry at room temperature. Reflectron MALDI-TOF spectra were acquired on a M@LDI (Micromass Wythenshawe, UK). The resulting peptide spectra were used to search with MassLynx ProteinProbe (Micromass Wythenshawe, UK) in a Fasta database or the sequence databases of the Mascot search engine (http://www.matrixscience.com).

Statistics. All statistical comparisons were performed using ANOVA. P<0.01, P<0.02 and P<0.05 were considered statistically significant.

### BRIEF DESCRIPTION OF THE DRAWINGS

FIG. 1

Direct infection of CD4⁺ T-lymphocytes in the presence of human milk. (A)

PBS or several dilutions of human milk from an uninfected mother were spiked with LAI (X4) and added to CD4 enriched T-lymphocytes. After a 2 hour incubation the CD4⁺ T-lymphocytes were washed and fresh medium was added. (B) LAI (X4) was incubated with a 1/2 dilution of human milk or PBS for 2 hours after which several dilutions were made and added to CD4⁺ T-lymphocytes. For both experiments the p24 concentration was determined on day 7. The asterisk represents a p-value of <0.05 in comparison to the PBS control.

FIG. 2

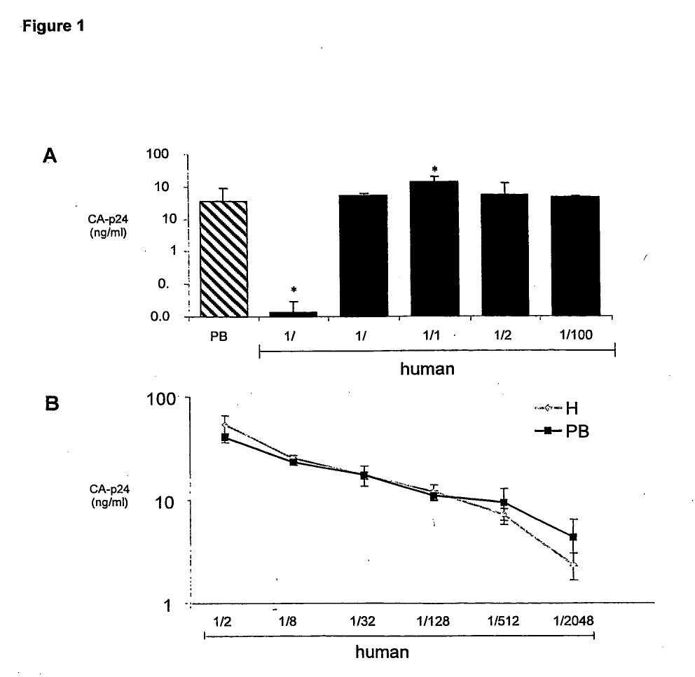

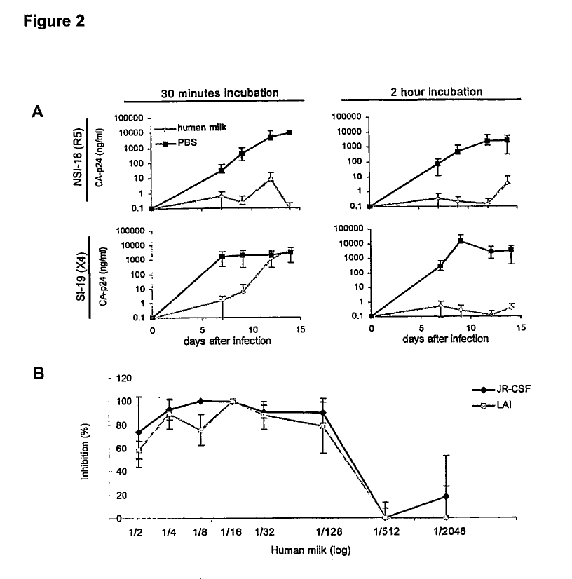

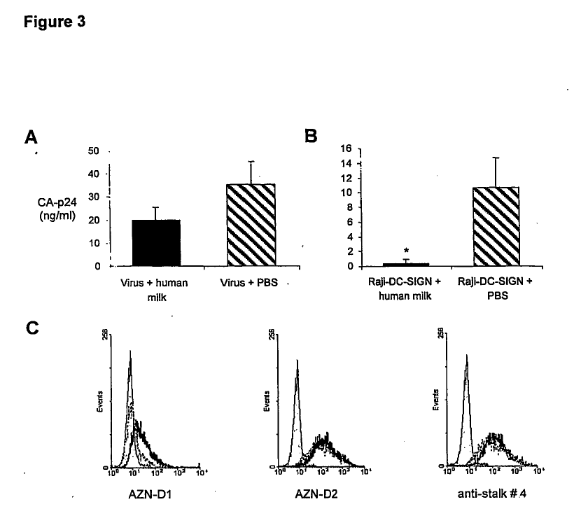

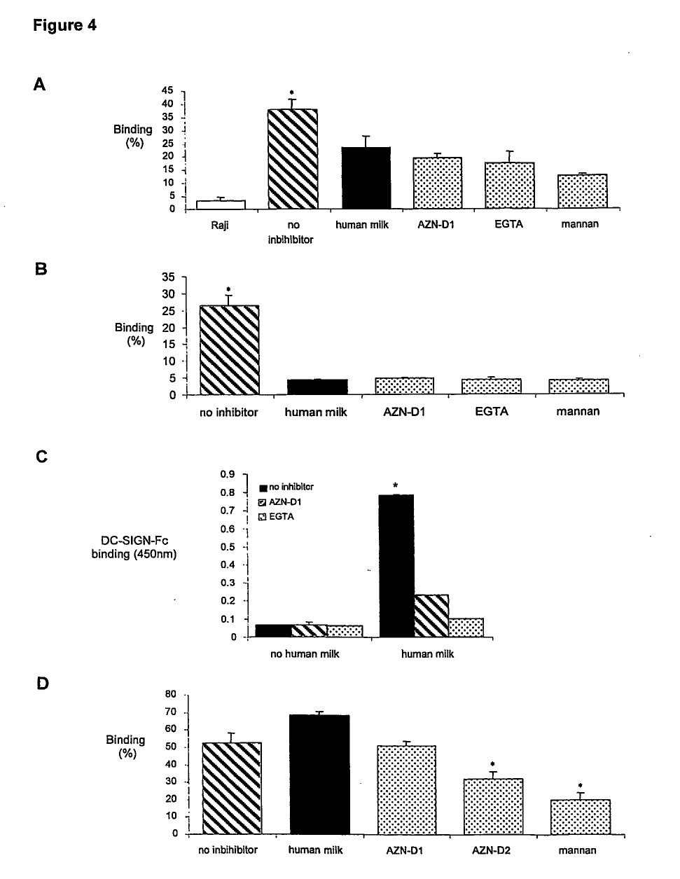

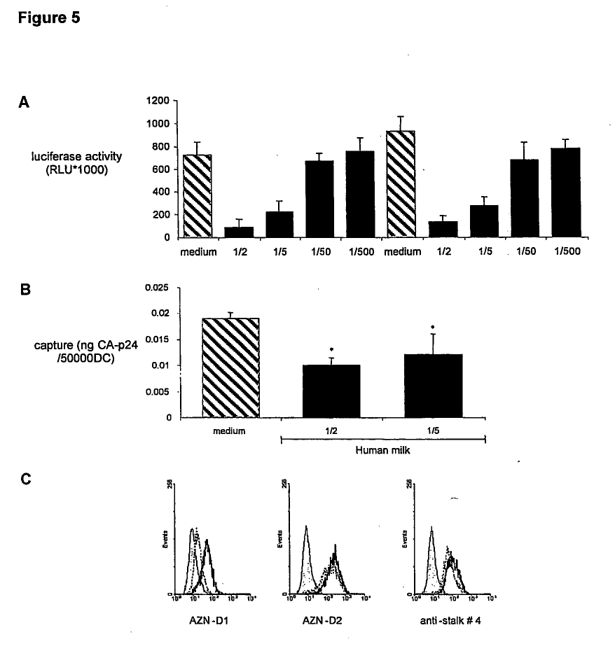

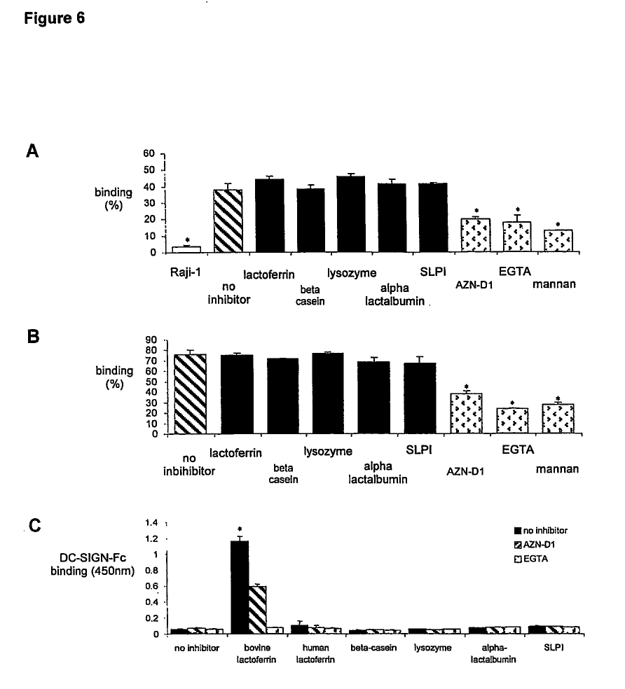

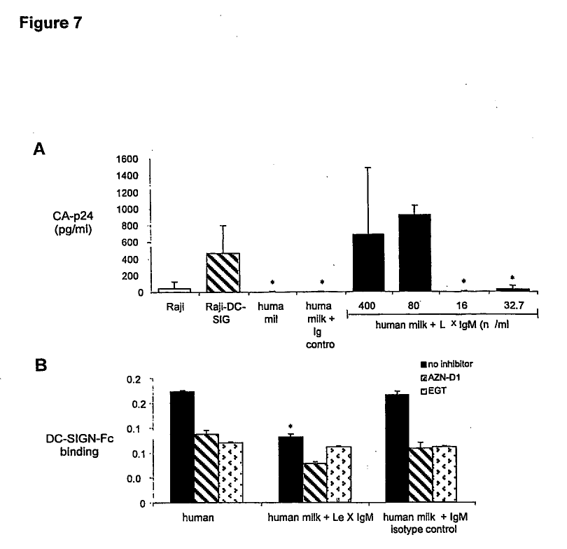

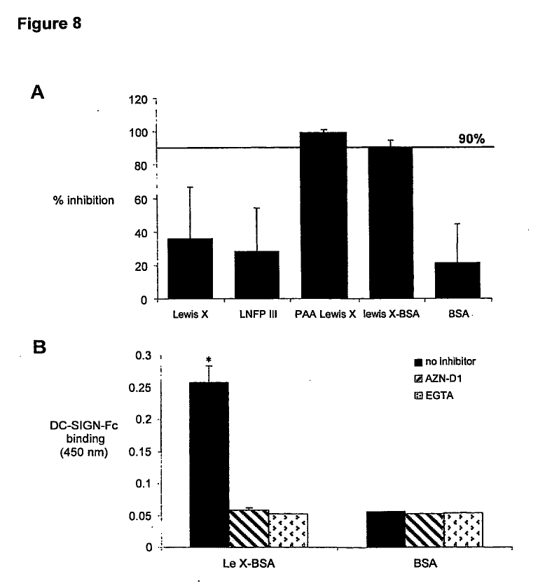

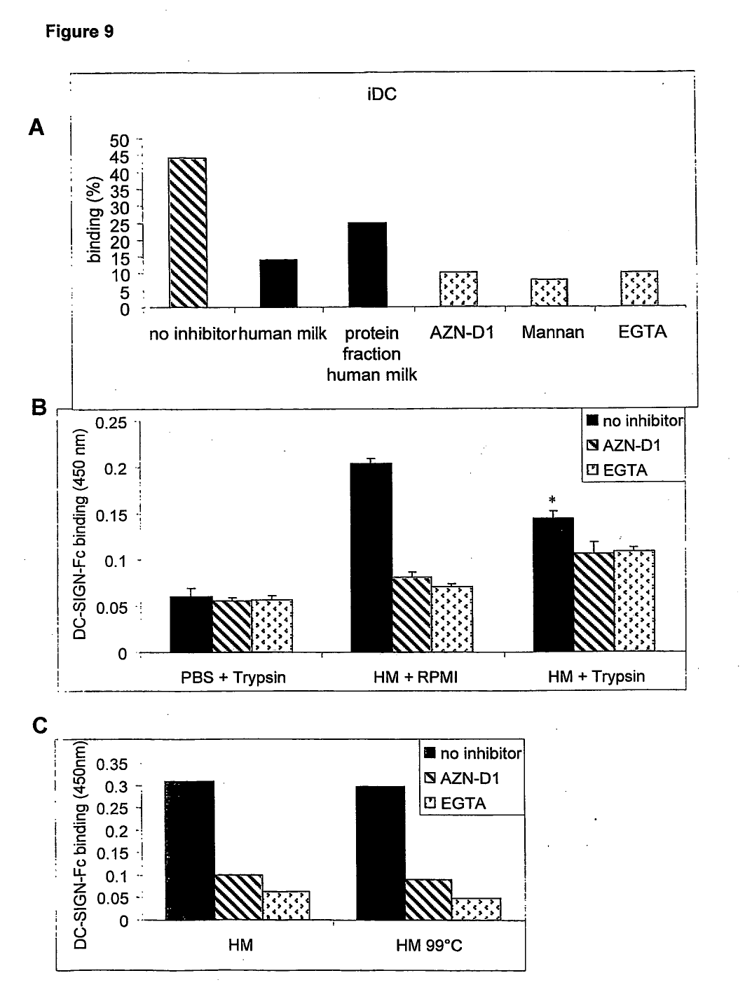

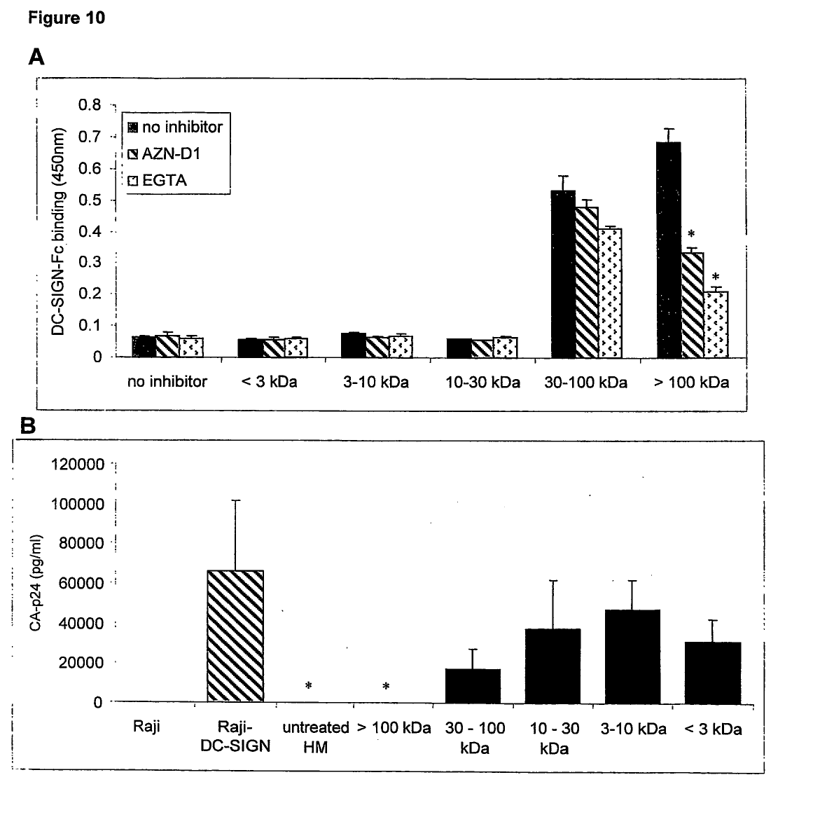

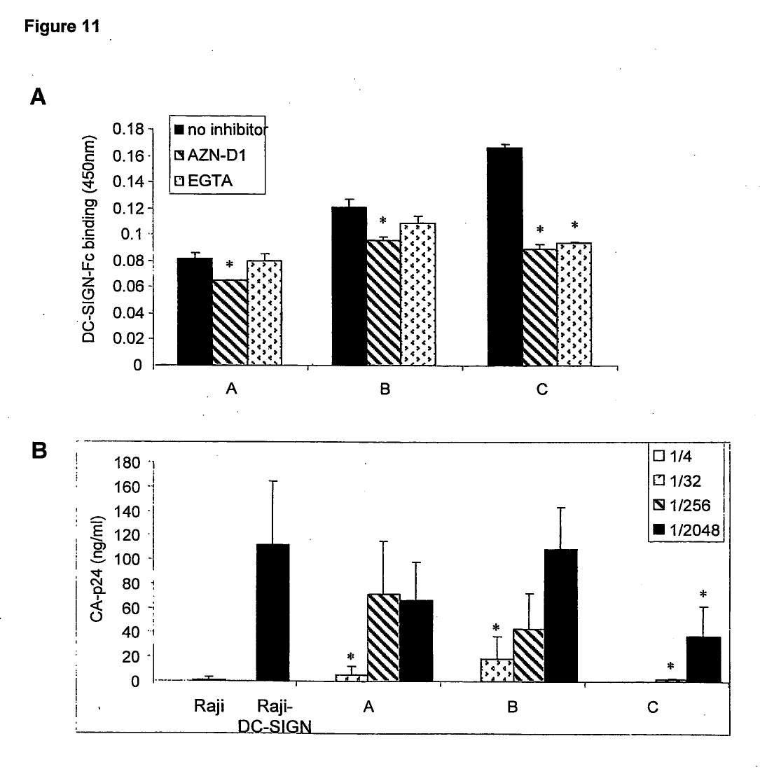

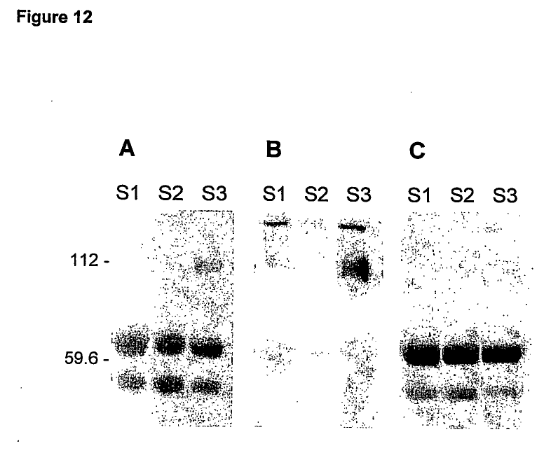

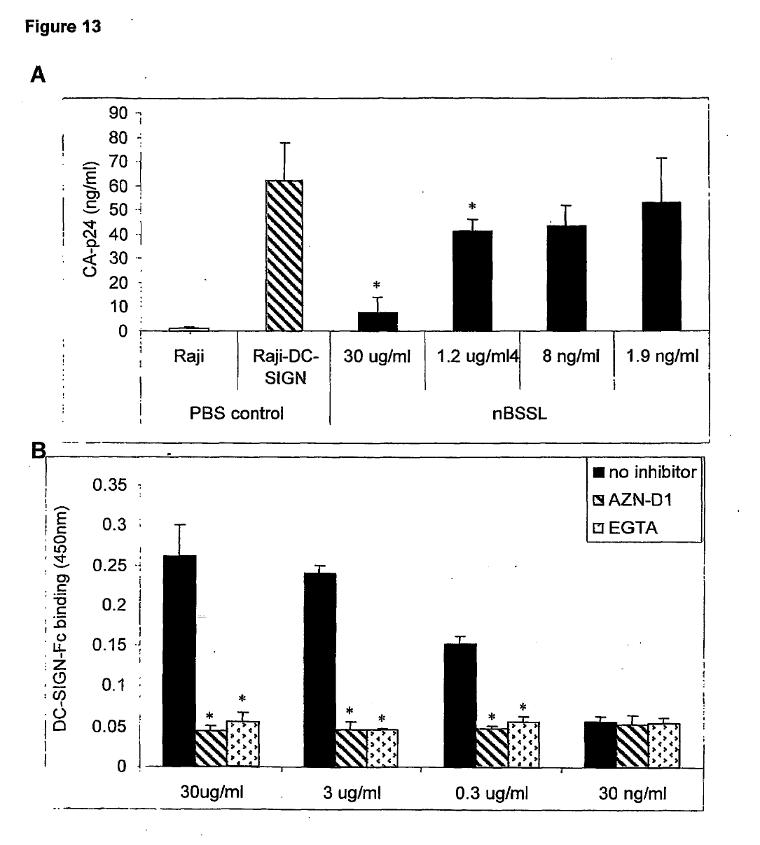

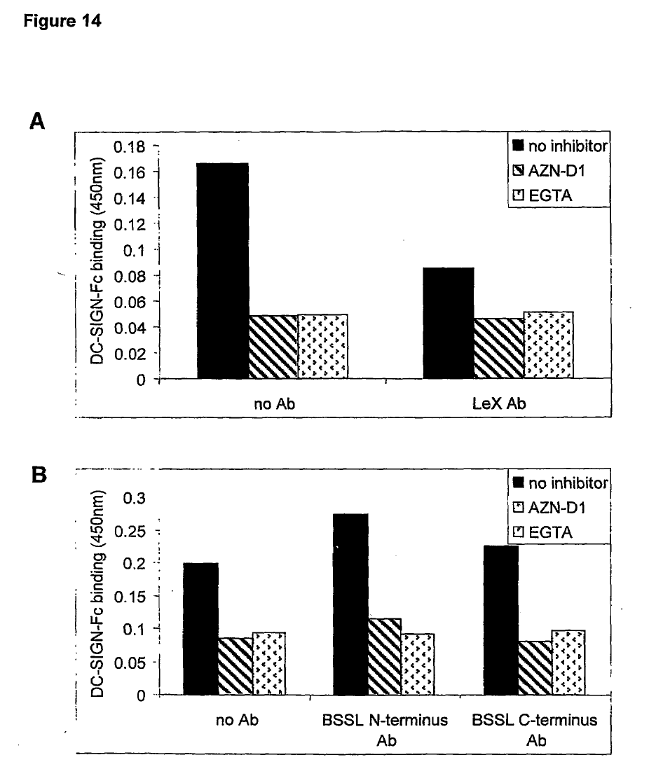

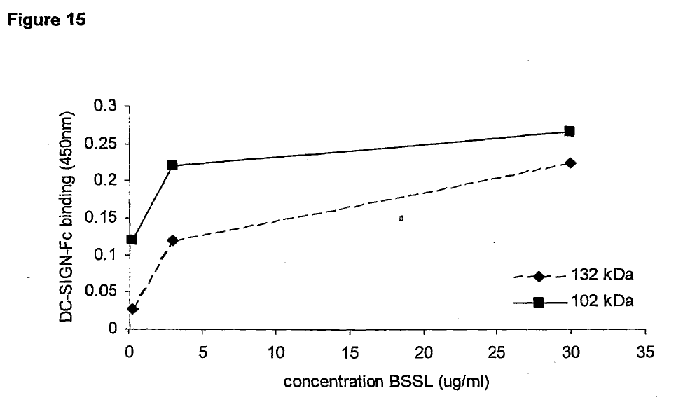

DC-SIGN dependent transfer of HIV-1 to CD4⁺ T-lymphocytes is inhibited in the presence of human milk. (A) A 1/2 dilution of human milk of an uninfected mother or PBS was spiked with primary isolates NSI-18 (R5) or SI-19 (X4) before addition to Raji-DC-SIGN cells. After an incubation of 30 minutes or 2 hours, the cells were washed and activated CD4+ T-lymphocytes were added. Viral replication was measured on days 7, 9, 12 and 14 after infection by determining p24 values using a standard ELISA. The depicted bars represent maximum and minimum p24 values. (B) PBS or serial dilutions of human milk were spiked with JR-CSF (R5) or LAI (X4) before addition of Raji-DC-SIGN cells, after an incubation of 2 hours the cells were washed with PBS and stimulated CD4+ T-lymphocytes were added. At day 7 p24 concentrations were determined by standard ELISA. Percentage of inhibition was determined in reference to the p24 concentration of the corresponding spiked PBS control.

FIG. 3

The human milk compound(s) interact with the DC-SIGN receptor which does not lead to DC-SIGN down-modulation. (A) Human milk (1/4) or PBS were incubated with LAI before addition of Raji-DC-SIGN cells. After incubation the cells were washed and CD4⁺ T-lymphocytes were added with p24 values measured on day 15 by standard ELISA (P>0.01). (B) Human milk (1/4) or PBS were incubated with Raji-DC-SIGN after which the cells were washed to remove unbound human milk components before addition of LAI. After incubation, the cells were washed again and CD4⁺ T-lymphocytes were added with the p24 values measured on day 15 by standard ELISA. The asterisk represents a P value of <0.01. (C) Raji-DC-SIGN cells were incubated with TSM or human milk (1/2) before the binding of AZN-D1, AZN-D2 and anti-stalk #4, DC-SIGN specific Ab, was determined. The filled histogram represents the isotype control, the black line represents the antibody binding without human milk pre-incubation and the dotted line represents the antibody binding after the cells were incubated with human milk.

FIG. 4

DC-SIGN-Fc binding ELISA and the gp120 bead adhesion assay demonstrate the interaction of the human milk compound(s) with DC-SIGN. (A & B) Raji-DC-SIGN cells or iDCs respectively were incubated with human milk (1/20) before addition of fluorescent gp120 coated beads. DC-SIGN positive cells and mock Raji cells were incubated with buffer as controls. To determine the specificity of the observed binding the cells were incubated with AZN-D1, EGTA and mannan before addition of the gp120 beads. The asterisk represents p<0.05 in comparison to the PBS control (C) Human milk (1/20) was coated before addition of DC-SIGN-Fc. The specificity of the observed binding was determined by the pre-incubation of DC-SIGN-Fc with AZN-D1 and EGTA. The asterisk represents p<0.01 in comparison to the AZN-D1 and EGTA control. (D) Raji cells expressing the L-SIGN receptor were incubated with buffer, human milk (1/20), AZN-D1, AZN-D2, or mannan before addition of the gp120 fluorescent beads. P<0.01 in comparison to the binding without an inhibitor.

FIG. 5

Human milk inhibits the transfer of HIV-1 by iDCs and mDCs. (A) Both iDCs and mDCs from the same donor were incubated with several dilutions of human milk for 30 minutes before addition of LAI (X4). After 2 hours the cells were washed and LuSIV cells were added, after 24 hours the LuSIV cells were washed and the luciferase activity was determined as described in materials and methods. (B) After an incubation of iDCs with human milk or PBS, the cells were washed and LAI was added. After an incubation of 2 hours the cells were washed again and CA-p24 was determined by ELISA, representing capture of HIV-1 by iDCs. The asterisk represents a P value of <0.05 in comparison to the corresponding control value for both experiments. (C) iDCs were incubated with TSM or human milk (1/2) before the binding of AZN-D1; AZN-D2 and anti-stalk #4, DC-SIGN specific antibodies, was determined. The filled histogram represents the isotype control, the black line represents the antibody binding without human milk pre-incubation and the dotted line represents the antibody binding after the cells were incubated with human milk.

FIG. 6

The major milk proteins are not responsible for the inhibitory effect of human milk. (A & B) Raji-DC-SIGN cells or iDCs respectively were incubated with the major milk proteins before addition of fluorescent gp120 coated beads, as control the cells were incubated with buffer. To determine the specificity of the observed binding the cells were incubated with AZN-D1, EGTA and mannan before addition of the gp120 beads. (C) The major milk proteins were coated on ELISA plates and DC-SIGN-Fc binding was measured. To determine the specificity of the observed binding, the DC-SIGN-Fc was pre-incubated with AZN-D1 and EGTA. The asterisk represents p<0.01 in comparison to both the AZN-D1 and EGTA control. In all experiments the major proteins were diluted to a 1/20 dilution of their physiological concentration in human milk.

FIG. 7

Incubation of human milk (1/20) with Le^{x} IgM Ab relieves the inhibitory properties of human milk on DC-SIGN mediated transfer of HIV-1 to CD4⁺ T-lymphocytes. (A) A 1/200 dilution of human milk was incubated with a serial dilution of Le^{x} IgM Ab before addition of the Raji-DC-SIGN cells. LAI was added and following a short incubation the cells were washed and activated CD4⁺ T-lymphocytes were added with p24 values determined at day 7. The asterisk represents a P value of <0.05 in comparison to the Raji-DC-SIGN control. (B) Human milk (1/200) was coated and pre-incubated with anti-LeX IgM antibody or an IgM isotype control before addition of DC-SIGN-FC to determine the binding. DC-SIGN-FC was pre-incubated with AZN-D1 and EGTA to determine the specificity of the observed binding. The asterisk represents p<0.01 in comparison to the human milk binding without antibody present.

FIG. 8

Multimeric and protein associated Le^{x} inhibits DC-SIGN mediated viral transfer. (A) Several Le^{x} containing compounds show a difference in their ability to block DC-SIGN dependent transfer of HIV-1 to CD4⁺ T-lymphocytes. (A) The Le^{x} trisaccharide, lacto-N-fucopentaose III (LNFP III), Le^{x} coupled to biotinylated polyacrylamide (PAA-Le^{x}), Le^{x}-BSA and BSA as a control were tested in the Raji-DC-SIGN culture transfer assay, all at a concentration of 10 μg/ml. The inhibition is depicted as a percentage of the Raji-DC-SIGN incubated with PBS. (B) Le^{x}-BSA and BSA as a control were coated before addition of DC-SIGN-FC to determine the binding. DC-SIGN-FC was pre-incubated with AZN-D1 and EGTA to determine the specificity of the observed binding. The asterisk represents p<0.01 in comparison to both the AZN-D1 and EGTA control.

FIG. 9

Biochemical analysis of DC-SIGN binding component in human milk. (A) iDCs were incubated with human milk or the protein fraction of the same milk sample before addition of fluorescent gp120 beads. AZN-D1, mannan and EGTA were used as controls to show DC-SIGN specific binding. (B) Human milk was incubated with trypsin or RPMI, the DC-SIGN-Fc binding capacity of the treated human milk was measured by ELISA. The DC-SIGN-Fc binding was controlled for by pre-incubation with AZN-D1 and EGTA. P<0.01 compared to the binding of untreated human milk (C) Human milk was heated at 99° C. for 10 minutes before determination of the DC-SIGN-Fc binding capacity. AZN-D1 and EGTA were used as controls to show DC-SIGN-Fc specific binding.

FIG. 10

The inhibitory component of human milk is present in the >100 kDa fraction of human milk. Size fractionation of human milk was performed by filter centrifugation. The different fractions were tested in the (A) DC-SIGN-Fc binding ELISA with AZN-D1 and EGTA controlling for DC-SIGN binding specificity, and (B) the Raji-DC-SIGN transfer culture assay. The Raji-DC-SIGN cells were incubated with the different human milk fractions and virus before addition of CD4⁺ T-lymphocytes. As a control Raji or Raji-DC-SIGN cells were incubated with PBS and virus before addition of CD4⁺ T-lymphocytes. CA-p24 production was measured at day 7 by standard ELISA. P<0.01 compared to the relevant control.

FIG. 11

Differences in DC-SIGN binding capacity of human milk samples from three mothers. (A) The DC-SIGN-Fc binding capacity was measured for three different human milk samples. Pre-incubation of DC-SIGN-Fc with AZN-D1 and EGTA controlled for DC-SIGN specific binding (P<0.01). (B) Different dilutions of the milk samples were tested in the Raji-DC-SIGN transfer culture assay. To control for infection, Raji or Raji DC-SIGN cells were incubated with PBS and virus before addition of CD4⁺ T-lymphocytes. CA-p24 production was determined on day 7 by standard ELISA. P<0.02 compared to the PBS control.

FIG. 12

Western blot and coomassie staining of three human milk samples with different DC-SIGN binding capacity. (A) western blot stained with Le^{x} specific antibody (B) western blot stained with DC-SIGN-Fc (C) coomassie stained SDS-PAGE gel.

FIG. 13

BSSL binds DC-SIGN and prevents transfer of HIV-1 to CD4⁺ lymphocytes. (A) Raji-DC-SIGN cells were incubated with different dilutions of BSSL isolated from human milk (S4) and virus before addition of CD4⁺ T-lymphocytes. As a control Raji and Raji-DC-SIGN cells were incubated with PBS. CA-p24 production was determined on day 7 by standard ELISA compared to the PBS control (P<0.05). (B) DC-SIGN-Fc binding capacity was determined by ELISA for different dilutions of BSSL isolated from human milk. To control for DC-SIGN specificity DC-SIGN-Fc was pre-incubated with AZN-D1 and EGTA compared to the relevant binding without inhibitor (P<0.01).

FIG. 14

DC-SIGN-Fc binding can be blocked with a Le^{x} specific antibody but not by BSSL N- or C-terminus directed antibodies. (A) and (B) BSSL was plated at a concentration of 0.3 μg/ml and before addition of DC-SIGN-Fc the BSSL was pre-incubated with the relevant antibody. DC-SIGN-Fc was also pre-incubated with AZN-D1 and EGTA to determine specificity of binding.

FIG. 15

Two BSSL isoforms isolated from the same human milk sample demonstrate a difference in DC-SIGN-Fc binding capacity. Different dilutions of the BSSL were plated and the DC-SIGN-Fc binding was determined by ELISA. To control for specificity of the binding the DC-SIGN-Fc was pre-incubated with AZN-D1 or EGTA. The highest binding of these controls was subtracted from the observed binding without an inhibitor.

### REFERENCES

Blackberg, L. & Hernell, O. (1981) Eur. J. Biochem 116, 221-225
Geijtenbeek, T. B., Torensma, R., van Vliet, S. J., van Duijnhoven, G. C., Adema, G. J., van Kooyk, Y., and Figdor, C. G. 2000 a. Identification of DC-SIGN, a novel dendritic cell-specific ICAM-3 receptor that supports primary immune responses. Cell 100:575-585
Geijtenbeek, T. B., Kwon, D. S., Torensma, R., van Vliet, S. J., van Duijnhoven, G. C., Middel, J., Cornelissen, I. L., Nottet, H. S., KewalRamani, V. N., Littman, D. R. et al. 2000 b. DC-SIGN, a dendritic cell-specific HIV-1-binding protein that enhances trans-infection of T cells. Cell 100:587-597
Geijtenbeek, T. B., van Duijnhoven, G. C., van Vliet, S. J., Krieger, E., Vriend, G., Figdor, C. G., and van Kooyk, Y. 2002. Identification of different binding sites in the dendritic cell-specific receptor DC-SIGN for ICAM-3 and HIV-1. J. Biol. Chem. 277:11314-11320
Groot, F., Geijtenbeek, T. B., Sanders, R. W., Baldwin, C. E., Sanchez-Hernandez, M., Floris, R., van Kooyk, Y., de Jong, E. C., and Berkhout, B. 2005. Lactoferrin prevents dendritic cell-mediated human immunodeficiency virus type 1 transmission by blocking the DC-SIGN—gp120 interaction. J Virol 79:3009-3015
Guo, Y., Feinberg, H., Conroy, E., Mitchell, D. A., Alvarez, R., Blixt, O., Taylor, M. E., Weis, W. I., and Drickamer, K. 2004. Structural basis for distinct ligand-binding and targeting properties of the receptors DC-SIGN and DC-SIGNR. Nat. Struct. Mol Biol 11:591-598
Halary, F., Amara, A., Lortat-Jacob, H., Messerle, M., Delaunay, T., Houles, C., Fieschi, F., Arenzana-Seisdedos, F., Moreau, J. F., and Dechanet-Merville, J. 2002. Human cytomegalovirus binding to DC-SIGN is required for dendritic cell infection and target cell trans-infection. Immunity 17:653-664
Pollakis, G., Kang, S., Kliphuis, A., Chalaby, M. I., Goudsmit, J., and Paxton, W. A. 2001. N-linked glycosylation of the HIV type-1 gp120 envelope glycoprotein as a major determinant of CCR5 and CXCR4 coreceptor utilization. J Biol Chem 276:13433-13441
Roos, J. W., Maughan, M. F., Liao, Z., Hildreth, J. E., and Clements, J. E. 2000. LuSIV cells: a reporter cell line for the detection and quantitation of a single cycle of HIV and SIV replication. Virol. 273:307-315
Shevchenko, A., Wilm, M., Vorm, O. & Mann, M. (1996) Anal. Chem. 68, 850-858
Wu, L., Martin, T. D., Carrington, M., and KewalRamani, V. N. 2004. Raji B cells, misidentified as THP-1 cells, stimulate DC-SIGN-mediated HIV transmission. Virol. 318:17-23

1. A method for influencing interaction between a DC-SIGN ligand and a DC-SIGN receptor that is exposed to said DC-SIGN ligand, comprising regulating interaction between said DC-SIGN receptor and a compound comprising at least one Lewis X sugar epitope, or a functional part, derivative and/or analogue thereof, and a non-saccharide moiety.

2. The method according to claim 1, wherein an interaction between said DC-SIGN receptor and said compound is regulated by regulating the amount and/or activity of said compound in an environment wherein said DC-SIGN receptor is exposed to said compound.

3. The method according to claim 1, wherein said non-saccharide moiety comprises a proteinaceous moiety and/or a polymer moiety.

4. The method according to claim 1, wherein said compound comprises polyacrylamide-Lewis X, bovine serum albumin-Lewis X and/or bile salt stimulated lipase.

5. The method according to claim 4, wherein said compound comprises a human bile salt stimulated lipase isoform of between 90 and 115 kDa.

6. The method according to claim 5, wherein said human bile salt stimulated lipase isoform has between 13-16 proline-rich 11-amino acid repeat units in its carboxyl-terminal region.

7-8. (canceled)

9. The method according to claim 1, wherein said DC-SIGN receptor is present on a dendritic cell.

10. The method according to claim 1, wherein binding of a DC-SIGN receptor to a DC-SIGN ligand is at least in part counteracted.

11. (canceled)

12. The method according to claim 1, comprising administering polyacrylamide-Lewis X, bovine serum albumin-Lewis X, bile salt stimulated lipase (BSSL), or a functional part, derivative and/or analogue thereof, and/or a composition comprising polyacrylamide-Lewis X, bovine serum albumin-Lewis X, bile salt stimulated lipase (BSSL) or a functional part, derivative and/or analogue thereof, to said DC-SIGN receptor.

13. The method according to claim 1, comprising upregulating expression of bile salt stimulated lipase (BSSL) in an individual comprising said DC-SIGN receptor.

14. A method for at least in part counteracting infection of an individual by a microorganism capable of binding a DC-SIGN receptor, and/or at least in part counteracting spread of a microorganism capable of binding a DC-SIGN receptor in an individual, the method comprising:
providing the individual with a compound comprising at least one Lewis X sugar epitope, or a functional part, derivative and/or analogue thereof, and a non-saccharide moiety, and/or increasing the amount and/or activity of a compound comprising at least one Lewis X sugar epitope, or a functional part, derivative and/or analogue thereof, and a non-saccharide moiety in the individual.

15-16. (canceled)

17. The method according to claim 14, wherein said compound comprises bile salt stimulated lipase.

18-25. (canceled)

26. A composition comprising a compound comprising at least one Lewis X sugar epitope, or a functional part, derivative and/or analogue thereof, and a non-saccharide moiety.

27. The composition according to claim 26, which is a pharmaceutical composition.

28. A food product that has been provided with an isolated, synthetic or recombinant compound comprising at least one Lewis X sugar epitope, or a functional part, derivative and/or analogue thereof, and a non-saccharide moiety.

29. The food product according to claim 28 comprising milk.

30. A method for regulating an immune response in an individual, the method comprising
influencing interaction between a DC-SIGN receptor of the individual and a DC-SIGN ligand with the method according to claim 1.

31. A method for determining whether a first individual is at a low risk of being infected by a microorganism capable of binding a DC-SIGN receptor, and/or at a low risk of transmitting a microorganism capable of binding a DC-SIGN receptor to a second individual, the method comprising
determining whether the first individual comprises a bile salt stimulated lipase (BSSL) isoform having between 13-16 proline-rich 11-amino acid repeat units in its carboxyl-terminal region and/or a bile salt stimulated lipase (BSSL) isoform of between 90 and 115 kDa.

32. A method for determining whether an individual which has been infected by a micro organism capable of binding a DC-SIGN receptor is at a low risk of a progressing disease course, the method comprising:
determining whether the individual comprises a bile salt stimulated lipase (BSSL) isoform having between 13-16 proline-rich 11-amino acid repeat units in its carboxyl-terminal region and/or a bile salt stimulated lipase (BSSL) isoform of between 90 and 115 kDa.

33. A method for producing a compound capable of being specifically targeted to a dendritic cell, the method comprising:
providing said compound with at least one Lewis X sugar epitope, or a functional part, derivative and/or analogue thereof, and a non-saccharide moiety.

34. The method according to claim 33, wherein said Lewis X sugar epitope, or a functional part, derivative and/or analogue thereof, and a non-saccharide moiety comprises polyacrylamide-Lewis X, bovine serum albumin-Lewis X and/or bile salt stimulated lipase (BSSL), or a functional part, derivative and/or analogue thereof.

35. The method according to claim 34, wherein BSSL comprises a human bile salt stimulated lipase isoform having between 13-16 proline-rich 11-amino acid repeat units in its carboxyl-terminal region and/or a human bile salt stimulated lipase isoform of between 90 and 115 kDa.

36. The method according to claim 33, wherein said compound comprises a toxic agent, a micro organism, a virus-like particle and/or a pathogen antigen.

37. A non-human animal comprising:
an exogenous nucleic acid sequence encoding bile salt stimulated lipase (BSSL) and/or a functional part, derivative and/or analogue thereof; or
a compound capable of enhancing expression and/or activity of BSSL or a functional part, derivative and/or analogue thereof.

38. (canceled)
